SEQUENCE LISTING (5 sequences)

SEQ ID NO: 1 - DNA, 39 bp, synthetic construct
  misc_feature 1..39: note = Catsup forward primer
  misc_feature 1..39: note = source = sequence is synthesized
  source 1..39: mol_type = other DNA
ctctgaatagggaattgggatggccaaacaagtggctga

SEQ ID NO: 2 - DNA, 43 bp, synthetic construct
  misc_feature 1..43: note = Catsup reverse primer
  misc_feature 1..43: note = source = sequence is synthesized
  source 1..43: mol_type = other DNA
ccgcagatctgttaacgtcacgtagaatcgagaccgaggagag

SEQ ID NO: 3 - DNA, 705 bp, synthetic construct
  misc_feature 1..705: note = UAS-RNAi-resistant Catsup
  source 1..705: mol_type = other DNA
acacggccattcccatgacatgtccatcggcttgtgggtgctgggcggcattatcgcgtttctgagcgtcgaaaagttggtgcgtatcctgaaaggaggccacggcggccatggacattcccacggcgcccccaaacccaagcccgtccccgccaaaaagaaaagcagcgataaggaggattccggcgacggcgataagcccgccaaacccgcgaaaattaaaagcaaaaagcccgaggccgaacccgagggagaggtcgaaatcagcggatatctgaacctggccgccgatttcgcccataattttacggacggattggcgattggagcgagctacctggccggaaattccatcggaatcgtcacgaccattaccatcttgttgcatgaggtcccgcacgaaatcggcgatttcgcgatcctgatcaaaagcggatgcagccgccgcaaagccatgctgttgcaactggtgaccgccctgggcgccctggccggaaccgccctggccctgttgggcgccggcggaggcgatggcagcgcgccctgggtgctgccgtttaccgcgggaggcttcatctatattgccaccgtcagcgtgttgcccgaattgctggaagaaagcaccaagttgaagcaaagcttgaaagagattttcgccttgctgaccggcgtagccctaatgatcgttatcgccaagttcgaggg

SEQ ID NO: 4 - protein, 449 aa, Drosophila melanogaster
MAKQVADYQCSKLQLYQKLALVVILGAVLFSLPALCAGQGNPSFKYSREANENFDPQKAPRAEHHHHHDHDHDHGHHHHGHDHDHDHDHGHDHGHHHHGHDHDHDHDHGHHHHGHDERHTKAKPDLDMSTIWLHSIGSTLLISAAPFVLLYIIPLDNSEAMKPRLKVLLAFASGGLLGDAFLHLIPHATHPHSHGEHGHDHGHDHHHHHDGEEHEHGHSHDMSIGLWVLGGIIAFLSVEKLVRILKGGHGGHGHSHGAPKPKPVPAKKKSSDKEDSGDGDKPAKPAKIKSKKPEAEPEGEVEISGYLNLAADFAHNFTDGLAIGASYLAGNSIGIVTTITILLHEVPHEIGDFAILIKSGCSRRKAMLLQLVTALGALAGTALALLGAGGGDGSAPWVLPFTAGGFIYIATVSVLPELLEESTKLKQSLKEIFALLTGVALMIVIAKFE

SEQ ID NO: 5 - protein, 469 aa, Homo sapiens
MARGLGAPHWVAVGLLTWATLGLLVAGLGGHDDLHDDLQEDFHGHSHRHSHEDFHHGHSHAHGHGHTHESIWHGHTHDHDHGHSHEDLHHGHSHGYSHESLYHRGHGHDHEHSHGGYGESGAPGIKQDLDAVTLWAYALGATVLISAAPFFVLFLIPVESNSPRHRSLLQILLSFASGGLLGDAFLHLIPHALEPHSHHTLEQPGHGHSHSGQGPILSVGLWVLSGIVAFLVVEKFVRHVKGGHGHSHGHGHAHSHTRGSHGHGRQERSTKEKQSSEEEEKETRGVQKRRGGSTVPKDGPVRPQNAEEEKRGLDLRVSGYLNLAADLAHNFTDGLAIGASFRGGRGLGILTTMTVLLHEVPHEVGDFAILVQSGCSKKQAMRLQLLTAVGALAGTACALLTEGGAVGSEIAGGAGPGWVLPFTAGGFIYVATVSVLPELLREASPLQSLLEVLGLLGGVIMMVLIAHLE

(19) United States
(12) Patent Application Publication
(10) Pub. No.: US 2025/0000945 A1
(43) Pub. Date: Jan. 2, 2025

(54) Suppression of Neurodegeneration with Zinc Transporter Protein 7

(71) Applicant: The Regents of the University of California, Oakland, CA (US)

(72) Inventors: Denise Montell, Santa Barbara, CA (US); Xiaoran (Sharon) Guo, Santa Barbara, CA (US); Diego Acosta Alvear, Santa Barbara, CA (US)

(21) Appl. No.: 18/690,533

(22) PCT Filed: Sep. 13, 2022
(86) PCT No.: PCT/US2022/076353

§ 371 Date: Mar. 8, 2024

Related U.S. Application Data
(60) Provisional application No. 63/243,590, filed on Sep. 13, 2021.

(51) Int. Cl. A61K 38/17; A61K 48/00; A61P 25/28

(52) U.S. Cl. CPC A61K 38/177; A61K 48/005; A61P 25/28

(57) ABSTRACT

Methods and compositions are provided for enhancing endoplasmic reticulum-associated degradation (ERAD) and suppressing pathological accumulation of misfolded proteins in cells by increasing expression or activity of zinc transporter protein 7 (ZIP7). Methods of treating a subject for a disorder associated with protein misfolding are also provided, including methods of gene therapy for expressing ZIP7 in vivo in effective amounts sufficient to suppress pathological accumulation of misfolded proteins.

### CROSS-REFERENCE TO RELATED APPLICATION

This application claims benefit under 35 U.S.C. § 119(e) of provisional application 63/243,590, filed Sep. 13, 2021, which application is hereby incorporated by reference in its entirety.

### STATEMENT REGARDING FEDERALLY SPONSORED RESEARCH OR DEVELOPMENT

This invention was made with government support under Grant No. RO1 GM046425 awarded by the National Institutes of Health. The government has certain rights in the invention.

### INCORPORATION BY REFERENCE OF SEQUENCE LISTING

A Sequence Listing is provided herewith as a Sequence Listing XML file, “UCSB-546WO_2022-758” created on Sep. 9, 2022 and having a size of 8,811 bytes. The contents of the Sequence Listing XML file are incorporated by reference herein in their entireties.

### INTRODUCTION

Collective cell migration has emerged as a key driver of normal organ development, wound repair, and tumor metastasis (Mishra et al., Development 146, (2019); Friedl & Mayor, Cold Spring Harb. Perspect. Biol. 9, (2017); Friedl & Gilmour, Nat. Rev. Mol. Cell Biol. 10, 445-457 (2009); Friedl et al., Nat. Cell Biol. 14, 777-783 (2012)). Border cell migration in the Drosophila ovary provides a powerful in vivo model of collective cell migration that is amenable to unbiased genetic screening. Drosophila ovaries are composed of ovarioles, which are strings of egg chambers progressing through 14 stages of development to mature eggs (FIG. 1A). Each egg chamber is composed of 16 germ cells, including 15 nurse cells and one oocyte, which are surrounded by epithelial follicle cells. During stage 9 (FIG. 1B), 4-8 border cells are specified at the anterior end of the egg chamber, delaminate from the follicular epithelium, and migrate posteriorly during developmental stage 9, reaching the anterior border of the oocyte by stage 10.

Genetic screens have yielded insights into the molecular mechanisms that specify which of the ˜850 follicle cells acquire the ability to migrate (Silver & Montell, Cell 107, 831-841 (2001); Bai & Montell, Development 129, 5377-5388 (2002)), the developmental timing of their migration (Jang et al., Nat. Cell Biol. 11, 569-579 (2009); Bai & Montell, Cell 103, 1047-1058 (2000)), direction sensing (McDonald et al., Development 130, 3469-3478 (2003); McDonald et al., Dev. Biol. 296, 94-103 (2006); Dai et al., Science 370(6519), 987-990 (2020)), and cytoskeletal dynamics (McDonald et al. (2003), supra; Murphy & Montell, J. Cell Biol. 133, 617-630 (1996); Kim et al., Genes Dev. 25, 730-741 (2011); Lee et al., Development 122, 409-418 (1996); Cai et al., Cell 157, 1146-1159 (2014); McDonald, Curr. Biol. 18, 1659-1667 (2008); Wang et al., Nat. Cell Biol. 12, 591-597 (2010); Duchek & Rorth, Science 291, 131-133 (2001); Fulga & Rorth, Nat. Cell Biol. 4, 715-719 (2002); Ramel, et al., Nat. Cell Biol. 15, 317-324 (2013); Assaker, et al., Proc Natl Acad Sci USA 107, 22558-22563 (2010)). Border cell studies continue to provide new biological insights (Dai et al., supra; Miao et al., Dev. Cell 54, 501-515.e9 (2020); Colombie et al., Dev. Biol. 423, 12-18 (2017); Chen et al., elife 9, e52979 (2020); Laflamme et al., J. Cell Biol. 198, 57-67 (2012); Ogienko et al., Int. J. Mol. Sci. 21, (2020); Berez et al., Front. Physiol. 11, 803 (2020); Wang et al., iScience 23, 101335 (2020); Wang, H., Guo, X., Wang, X., Wang, X. & Chen, J. Supracellular Actomyosin Mediates Cell-Cell Communication and Shapes Collective Migratory Morphology. iScience 23, 101204 (2020); Fox et al., Mol. Biol. Cell 31, 1584-1594 (2020); Plutoni et al., Nat. Commun. 10, 3940 (2019); Zeledon et al., Cell Rep. 28, 3238-3248.e3 (2019); Lamb et al., Dev. Dyn. 249, 961-982 (2020); Ghiglione, C., Jouandin, P., Cerezo, D. & Noselli, S. The Drosophila insulin pathway controls Profilin expression and dynamic actin-rich protrusions during collective cell migration. Development 145, dev161117 (2018); Sharma et al., Development 145, (2018)). The gene Catsup was identified both in a large-scale ethyl methanesulfonate mutagenesis screen for border cell migration defects in mosaic clones (Liu & Montell, Development 126, 1869-1878 (1999); and in a whole-genome expression profile of border cells (Wang et al., Dev. Cell 10, 483-495 (2006)).

The name Catsup is an abbreviation of “Catecholamines up”, loss of which increases synthesis of aromatic amines including neurotransmitters such as epinephrine and dopamine (Stathakis et al., Genetics 153, 361-382 (1999)). Catsup is required for Drosophila tracheal morphogenesis, and in this context, it directly binds and inhibits the Drosophila homolog of tyrosine hydroxylase Ple to limit dopamine synthesis (Hsouna et al., Dev. Biol. 308, 30-43 (2007)). In contrast, in wing imaginal disc cells, Catsup regulates Notch and EGFR abundance and localization (Groth et al., Development 140, 3018-3027 (2013)).

Catsup shares 62% similarity and 53% identity with its mammalian homolog zinc transporter protein 7 (ZIP7), also known as SLC39A7 or HKE4 (Groth et al., supra), a member of one of the two major families of Zn²⁺ transporters (Kambe, Zinc Transport: Regulation in Encyclopedia of inorganic and bioinorganic chemistry (ed. Scott, R. A.) 1-9 (John Wiley & Sons, Ltd, 2011). ZIP7 is located within the endomembrane system including the ER where it transports Zn²⁺ to the cytosol (Taylor et al., Biochem. J. 377, 131-139 (2004)). Zn²⁺ is a necessary trace element vital for many proteins to function, and Zn²⁺ homeostasis is carefully maintained by 24 Zn²⁺ transporters in humans, 14 of which are ZIPs⁴³. ZIP7 is conserved throughout eukaryotes, and its loss causes ER stress in organisms as diverse as yeast, plants, and animals (Nguyen et al., Biosci. Biotechnol. Biochem. 77, 1337-1339 (2013); Tan et al., Transgenic Res. 24, 109-122 (2015); Zhang et al., Int. J. Mol. Sci. 15, 20413-20433 (2014); Adulcikas et al., Comput. Biol. Med. 100, 196-202 (2018); Tuncay et al., Diabetes 66, 1346-1358 (2017); Fauster et al., Cell Death Differ. 26, 1138-1155 (2019)). However, the relationships between ER stress, Zn²⁺ transport, Notch and EGFR localization and activity, and cell motility remain to be clarified.

### SUMMARY

Methods and compositions are provided for enhancing endoplasmic reticulum-associated degradation (ERAD) and suppressing pathological accumulation of misfolded proteins in cells by increasing expression or activity of ZIP7. Methods of treating a subject for a disorder associated with protein misfolding are also provided, including methods of gene therapy for expressing ZIP7 in vivo in effective amounts sufficient to suppress pathological accumulation of misfolded proteins.

In one aspect, a method of treating a subject for a disorder associated with protein misfolding is provided, the method comprising administering to the subject a therapeutically effective amount of a zinc transporter protein 7 (ZIP7).

Disorders associated with protein misfolding that can be treated by the methods described herein include any condition resulting in protein misfolding and/or protein aggregation, wherein misfolded proteins and/or protein aggregates accumulate within the endoplasmic reticulum (ER) where they cause ER stress and inhibit ER-associated degradation (ERAD). Disorders associated with protein misfolding include, but are not limited to, retinitis pigmentosa and neurodegenerative diseases such as Huntington's disease, Alzheimer's disease, Parkinson's disease, frontotemporal dementia, amyotrophic lateral sclerosis (ALS), dentatorubropallidoluysian atrophy (DRPLA), spinal and bulbar muscular atrophy (SBMA) also known as Kennedy's disease, multiple system atrophy, and spinocerebellar ataxia (SCA).

In certain embodiments, the ZIP7 protein comprises an amino acid sequence having at least about 80-100% sequence identity to the sequence of SEQ ID NO:5, including any percent identity within this range, such as 81, 82, 83, 84, 85, 86, 87, 88, 89, 90, 91, 92, 93, 94, 95, 96, 97, 98, or 99% sequence identity thereto.

In certain embodiments, the ZIP7 protein is administered locally into an eye or the brain of the subject. In some embodiments, the ZIP7 protein is administered locally into a photoreceptor of the eye.

In certain embodiments, the ZIP7 protein is administered according to a daily dosing regimen or intermittently.

In certain embodiments, the ZIP7 protein is provided by an expression vector such as a viral vector.

In certain embodiments, the misfolded protein is a variant rhodopsin (e.g., Rh1G69D), Vap33, or amyloid β42.

In another aspect, a method of providing a subject with ZIP7 to suppress pathological accumulation of a misfolded protein is provided, the method comprising introducing an expression vector comprising a promoter operably linked to a coding sequence encoding the ZIP7 into a cell, wherein the cell expresses the ZIP in vivo in the subject in an effective amount sufficient to suppress the pathological accumulation of the misfolded protein in the cell.

In certain embodiments, the cell is a retina cell or a brain cell. In some embodiments, the retina cell is a photoreceptor cell.

In certain embodiments, the misfolded protein is a misfolded rhodopsin protein.

In certain embodiments, the expression vector is introduced into the cell ex vivo or in vivo.

In another aspect, a method of treating a subject for a disorder associated with protein misfolding is provided, the method comprising administering an expression vector comprising a promoter operably linked to a nucleotide sequence encoding ZIP7 to the subject, wherein the ZIP7 is expressed in vivo in the subject in a therapeutically effective amount sufficient to suppress pathological accumulation of the misfolded protein.

In another aspect, a method of enhancing endoplasmic reticulum (ER)-associated degradation (ERAD) or proteosome-associated degradation in a cell is provided, the method comprising increasing expression or activity of ZIP7 in the cell.

In certain embodiments, the cell is a retina cell. In some embodiments, the retina cell is a photoreceptor cell.

In certain embodiments, the expression of ZIP7 is increased sufficiently to increase degradation of a misfolded rhodopsin protein and suppress accumulation of the misfolded rhodopsin protein in the retina cell.

In certain embodiments, increasing expression of ZIP7 comprises transfecting the cell with a recombinant polynucleotide comprising a coding sequence encoding ZIP7. In some embodiments, the recombinant polynucleotide further comprises a viral vector comprising a promoter operably linked to the coding sequence encoding the ZIP7. In some embodiments, the coding sequence encoding the ZIP7 is integrated into a chromosomal locus of the transfected cell, wherein an endogenous promoter is operably linked to the integrated coding sequence encoding the ZIP7 at the chromosomal locus.

In another aspect, a method of suppressing accumulation of a misfolded protein in an organ, cell, or tissue of a subject is provided, the method comprising increasing expression or activity of ZIP7 in the organ, cell, or tissue.

In certain embodiments, the organ is an eye or a brain.

In certain embodiments, the tissue is neural tissue.

In certain embodiments, the tissue is retina tissue.

In certain embodiments, the cell is a retina cell. In some embodiments, the retina cell is a photoreceptor cell.

In certain embodiments, the misfolded protein is a rhodopsin protein.

In certain embodiments, the subject has retinitis pigmentosa.

In certain embodiments, the disorder associated with protein misfolding is retinitis pigmentosa, Huntington's disease, Alzheimer's disease, Parkinson's disease, amyotrophic lateral sclerosis, or frontotemporal dementia.

In certain embodiments, the expression of ZIP7 is increased sufficiently to decrease pathological accumulation of the misfolded protein and increase cell survival.

In certain embodiments, the expression of ZIP7 is increased by providing a recombinant polynucleotide comprising a coding sequence encoding the ZIP7 to the organ, cell, or tissue, wherein the ZIP7 is expressed in the organ, cell, or tissue.

In certain embodiments, the recombinant polynucleotide further comprises a viral vector comprising a promoter operably linked to the coding sequence encoding the ZIP7.

In certain embodiments, the coding sequence encoding the ZIP7 is integrated into a chromosomal locus. In some embodiments, an endogenous promoter is operably linked to the integrated coding sequence encoding the ZIP7 at the chromosomal locus.

In another aspect, a composition for use in a method of treating a disorder associated with protein misfolding is provided, the composition comprising ZIP7 or an expression vector comprising a promoter operably linked to a coding sequence encoding ZIP7. In some embodiments, the composition further comprises a pharmaceutically acceptable excipient.

### BRIEF DESCRIPTION OF THE DRAWINGS

FIGS. 1A-1T. Catsup expression and subcellular localization in the Drosophila ovary. (FIGS. 1A, 1B) Developing egg chambers from the germarium to developmental stage 10, expressing Catsup::GFP and stained for DNA with Hoechst (blue) and F-actin with phalloidin (magenta). Border cells (white arrowheads) migrate during stage 9 (FIG. 1B) and complete their migration by stage 10 (FIG. 1A). (A′, B′) The single Catsup::GFP channel (grayscale). (FIGS. 1C-1G) High magnification of a border cell cluster showing the localization of overexpressed CatsupV5 (yellow), anti-PDI staining for ER (green), phalloidin (magenta), and Hoechst (blue). (FIGS. 1H-1J) 2-dimensional intensity histograms for two selected channels showing colocalization of CatsupV5 relative to ER, F-actin, and DNA. The colocalization regression Pearson's coefficient is displayed in the upper right corner. (FIG. 1K) Comparison of Pearson's coefficients (average of 4 border cell clusters). **P value<0.01. (FIGS. 1L-1P) High magnification of a border cell cluster showing the localization of tagged Catsup::GFP expressed under endogenous regulatory sequences (green), ER (PDI, yellow), F-actin (phalloidin, magenta) and nuclear DNA (Hoechst, blue). (FIGS. 1Q-1T) 2-dimensional intensity histograms for two selected channels showing the colocalization and the Pearson's coefficient for Catsup::GFP relative to ER, F-actin, nuclei, as well as ER relative to F-actin. Scale bars, 20 μm.

FIGS. 2A-2J. Border cells require Catsup for normal migration. (FIGS. 2A, 2B) Confocal micrographs of stage 10 egg chambers in which fruitlessGal4 drives expression UAS-Catsup RNAi and (FIG. 2A) UAS-GFPnls or (FIG. 2B) UAS-CatsupV5 in outer, migratory border cells. (FIGS. 2C-2D) Catsup::GFP expression in control border cells (FIG. 2C) or knocked down by c306Gal4>CatsupRNAi (FIG. 2D). (FIG. 2E) Quantification of incomplete migration at stage 10 in fruitlessGal4 (blue) and c306Gal4 (magenta) driving the indicated transgenes. Experiments were independently replicated 3 times. (FIG. 2F) An egg chamber with GFP-negative (homozygous Catsup mutant) cells. Both polar cells (p) and two border cells (b) are mutant. (FIG. 2G) An egg chamber in which all outer border cells are GFP-negative (homozygous Catsup mutant). (FIG. 2H) Migration distance expressed as a percentage of the migration path for mosaic border cell clusters as a function of the proportion of homozygous mutant cells in each cluster. (FIG. 2I) High magnification view showing the spatial distribution of Castup⁺ (GFP⁺) and Catsup^{-/-} (GFP^{-/-}) cells in a migrating cluster. (FIG. 2J) Quantification of the percentage of Catsup⁺ vs Catsup^{-/-} border cells in the front, side, or back of the border cell cluster showing that Catsup^{-/-} cells are more likely to occupy a rear position. “p” indicated polar cells, “b” indicated border cells, green labels control cells, yellow labels mutant cells. **P≤0.01, ***P≤0.001, ****P≤0.0001. Scale bars, 20 μm.

FIGS. 3A-31. Altered Notch and EGFR abundance and localization in cells expressing CatsupRNAi. (FIGS. 3A, 3B) Differential interference contrast images of stage 10 egg chambers stained for Ple (magenta) in the w¹¹¹⁸ control (FIG. 3A) or an egg chamber expressing UAS-Ple with c306Gal4 (FIG. 3B). (FIG. 3C) A dissected fly brain stained for endogenous Ple: dopaminergic neurons are Ple-positive. (FIGS. 3D-3E) CatsupRNAi-expressing clones (GFP-positive, green) accumulate intracellular Notch protein in epithelial follicle cells (FIG. 3D) and border cells (FIG. 3E) relative to neighboring wild type cells. (FIG. 3F) CatsupRNAi-expressing border cells (GFP-positive, green) show decreased Notch signaling shown by the Notch-responsive-element driving RFP (white) relative to neighboring wild type cells. (FIGS. 3G-3H) Accumulation of EGFR (magenta) in CatsupRNAi-expressing border cells (FIG. 3G) and epithelial follicle cells (FIG. 3H). (FIG. 31) c306Gal4>CatsupRNAi reduces Catsup::GFP expression but does not cause E-cadherin (magenta) intracellular accumulation. Scale bars, 20 μm.

FIGS. 4A-4L. ER stress in Catsup mutant border cells. (FIG. 4A) A mosaic border cell cluster composed of a mixture of control cells (RFP-positive, magenta which can be Catsup^{+/+} or Catsup^{-/+}) and homozygous Catsup mutant cells (RFP-negative, outlined). Polar cells (p) express a higher level of RFP compared to outer border cells. Xbp1::EGFP (green), a marker for ER stress. (FIG. 4B) Mosaic follicle cell clones expressing CatsupRNAi and GFPnls caused ER expansion shown by PDI (magenta). (FIG. 4C) Mosaic clone expressing CatsupRNAi and CatsupV5 and RFP (magenta). (FIG. 4D) Expression of a misfolded rhodopsin protein Rh1^{G69D} (magenta) in border cells induced ER stress (Xbp1::EGFP in green) and blocked border cell migration. (FIG. 4E) Co-expressing CatsupV5 reduced the Rh1^{G69D} protein level (magenta) and Xbp1::EGFP and rescued migration. (FIG. 4F) The percentage of border cells expressing RH1^{G69D} that are Xbp1 positive, in the absence (blue dots) or presence (pink dots) of CatsupV5 rescue. mIFR is a control, irrelevant fluorescent protein. (FIG. 4G) CatsupV5 rescue of RH1^{G69D} migration defect. (FIGS. 4H-4L) Mosaic clones expressing RH1^{G69D}; GFP shows comparable Notch intensity in epithelial clones (FIG. 4H) and a border cell clone (I, I′); EGFR intensity in epithelial clones (FIG. 4J) and a border cell clone (FIG. 4K); The Notch responsive element reporter shows Notch transcriptional activity (white) in wild type but not Catsup RNAi expressing border cells (FIG. 4L). **P≤0.01, ****P≤0.0001. Scale bars, 20 μm.

FIGS. 5A-50. Point mutations suggest requirement for Zn²⁺ transport in ER homeostasis and cell motility. (FIG. 5A) Schematic representation of transmembrane domains and topology for Catsup. Point mutations H183A and H187A reside within the second transmembrane domain while H315A and H344A are within the HELP domain required for Zn²⁺ transport. (FIGS. 5B-5E) Expression and co-localization of V5-tagged, RNAo-resistant Catsup mutants with the ER marker PDI (green) in border cells. (FIG. 5F) Quantification of incomplete migration at stage 10 in egg chambers expressing CatsupRNAi together with the indicated mutant forms. Experiments were independently repeated three times. (FIGS. 5G-5N) Mosaic expression of CatsupRNAi together with the indicated mutant forms of Catsup marked by RFPnls (magenta) and stained for Notch or EGFR in green, as indicated. Scale bars, 20 μm. (FIG. 50) Model for the function of Catsup/ZIP7.

FIGS. 6A-6B. ZIP7 prevents Rh1G69D-induced photoreceptor cell death, which causes a rough eye. (FIG. 6A) Percentage of flies having the Rh1^{G69D} genotype that develop rough eye in presence and absence of ZIP7 expression. (FIG. 6B) Comparison of eye morphology of flies having the Rh1^{G69D} genotype in presence and absence of ZIP7 expression.

FIG. 7. Overexpression of ZIP7 mitigates ER stress and neuronal cell death in protein aggregation diseases.

FIG. 8. Screening aggregate prone proteins for Zip7 rescue.

FIG. 9. ZIP7 overexpression suppresses neurodegeneration due to expression of Vap33.

FIG. 10. ZIP7 overexpression suppresses neurodegeneration due to expression of amyloid β42.

FIG. 11. Schematic showing the role of Zn²⁺. The ubiquitin ligases are RING finger domain proteins that require Zn²⁺. Many protein components of the proteasome require Zn²⁺. ZIP7 overexpression enhances the activity of the proteasome, even in the presence of a proteasome inhibitor.

FIGS. 12A-12C. Overexpressing ZIP7 decreases ubiquitinated protein levels. (FIG. 12A) Egg chambers with and without ZIP7 overexpression (OE) were incubated with media for 5 hours. (FIG. 12B) Egg chambers with and without ZIP7 overexpression (OE) were treated with 10 μM MG132 proteasome inhibitor for 5 hours. (FIG. 12C) Comparison of FK2/DNA ratio egg for chambers incubated with media or 10 μM MG132 proteasome inhibitor for 5 hours with or without ZIP7 overexpression.

### DETAILED DESCRIPTION

Methods and compositions are provided for enhancing endoplasmic reticulum-associated degradation (ERAD) and suppressing pathological accumulation of misfolded proteins in cells by increasing expression or activity of zinc transporter protein 7 (ZIP7). Methods of treating a subject for a disorder associated with protein misfolding are also provided, including methods of gene therapy for expressing ZIP7 in vivo in effective amounts sufficient to suppress pathological accumulation of misfolded proteins.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a protein” includes a plurality of such proteins and reference to “the protein” includes reference to one or more proteins and equivalents thereof, e.g., polypeptides and peptides, known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The term “disorder associated with protein misfolding” is used herein to refer to any condition resulting in protein misfolding and/or protein aggregation, wherein misfolded proteins and/or protein aggregates accumulate within the endoplasmic reticulum (ER) where they cause ER stress and inhibit ER-associated degradation (ERAD). Aggregation of misfolded proteins may further cause cellular dysfunction, loss of synaptic connections, neuron death, eye damage, and/or brain damage. Disorders associated with protein misfolding include, but are not limited to, retinitis pigmentosa and neurodegenerative diseases such as Huntington's disease, Alzheimer's disease, Parkinson's disease, frontotemporal dementia, amyotrophic lateral sclerosis (ALS), dentatorubropallidoluysian atrophy (DRPLA), spinal and bulbar muscular atrophy (SBMA) also known as Kennedy's disease, multiple system atrophy, and spinocerebellar ataxia (SCA).

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with a disorder associated with protein misfolding) as well as those in which prevention is desired (e.g., those with increased susceptibility or a genetic predisposition to developing a disorder associated with protein misfolding).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “subject” as used herein refers to a patient in need of the treatments disclosed herein. The patient may be a mammal, such as, a rodent, a feline, a canine, a primate, or a human, e.g., a child, an adolescent, an adult, such as a young, middle-aged, or elderly human. The patient may have been diagnosed as having a disorder associated with protein misfolding or may be suspected of suffering from a disorder associated with protein misfolding.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “survival” as used herein means the time from the start of treatment to the time of death.

By “therapeutically effective dose or amount” of a ZIP7 protein or a recombinant polynucleotide comprising a coding sequence encoding a ZIP7 protein is intended an amount that, when administered, as described herein, brings about a positive therapeutic response, such as improved recovery from a disorder associated with protein misfolding. Improved recovery may include improved ERAD function resulting in increased degradation of misfolded proteins and reduced formation of protein aggregates, restored neuronal function, improved cognition, improved memory, and/or increased survival. In the case of retinitis pigmentosa, a therapeutically effective dose or amount of a ZIP7 protein or a recombinant polynucleotide comprising a coding sequence encoding a ZIP7 protein may reduce accumulation of misfolded rhodopsin and rhodopsin protein aggregates in the ER and photoreceptors in the retina of the eye, reduce ER stress, improve survival of photoreceptor cells, and prevent or delay eye damage and loss of vision. In the case of an amyloid beta aggregation-associated disease (e.g., Alzheimer's disease), a therapeutically effective dose or amount” of a ZIP7 protein may reduce amyloid beta aggregation and reduce formation of amyloid plaques in the brain. Additionally, a therapeutically effective dose or amount may retard loss of cerebellar Purkinje neurons and loss of brain cells. In the case of an alpha-synuclein aggregation-associated disease or synucleinopathy (e.g., Parkinson's disease), a therapeutically effective dose or amount” of a ZIP protein may reduce aggregation of alpha-synuclein. Additionally, a therapeutically effective dose or amount may reduce accumulation of aggregates of alpha-synuclein in neurons, nerve fibers, and/or glial cells and reduce formation of Lewy bodies.

The terms “peptide,” “oligopeptide” and “polypeptide” refer to any compound comprising naturally occurring or synthetic amino acid polymers or amino acid-like molecules including but not limited to compounds comprising amino and/or imino molecules. No particular size is implied by use of the terms “peptide,” “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and U.S. Pat. No. 5,977,301; Nguyen et al. (2000) Chem Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. Preferably, the polypeptide is between about 3 and 100 residues in length. Generally, one skilled in art can easily select the maximum length in view of the teachings herein. Further, peptides and polypeptides, as described herein, for example synthetic peptides, may include additional molecules such as labels or other chemical moieties.

Thus, references to polypeptides or peptides also include derivatives of the amino acid sequences of the invention including one or more non-naturally occurring amino acids. A first polypeptide or peptide is “derived from” a second polypeptide or peptide if it is (i) encoded by a first polynucleotide derived from a second polynucleotide encoding the second polypeptide or peptide, or (ii) displays sequence identity to the second polypeptide or peptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include postexpression modifications of the polypeptide or peptide, for example, glycosylation, acetylation, phosphorylation, and the like.

Amino acid derivatives can also include modifications to the native sequence, such as deletions, additions and substitutions (generally conservative in nature), so long as the protein (or fragment thereof) maintains the desired activity (e.g., ZIP7 biological activity, ability to improve ERAD function, increase degradation of misfolded proteins, reduce aggregation of misfolded proteins, and/or reduce ER stress). These modifications may be deliberate, as through site-directed mutagenesis, or may be accidental, such as through mutations of hosts that produce the proteins or errors due to PCR amplification. Furthermore, modifications may be made that have one or more of the following effects: increasing ability to enhance ERAD, increase degradation of misfolded proteins and/or suppress protein aggregation, or facilitating purification, delivery, or cell processing. Proteins or biologically active fragments thereof can be made recombinantly, synthetically, or in tissue culture.

The term “zinc transporter protein 7” or “ZIP7” as used herein encompasses all forms of ZIP7 and also includes biologically active fragments, variants, analogs, and derivatives thereof that retain biological activity (e.g., ability to improve ERAD function, increase degradation of misfolded proteins, and/or suppress protein aggregation).

A ZIP7 polynucleotide, nucleic acid, oligonucleotide, protein, polypeptide, or peptide refers to a molecule derived from any source. The molecule need not be physically derived from an organism, but may be synthetically or recombinantly produced. A number of ZIP7 nucleic acid and protein sequences are known. A representative sequence of a ZIP7 protein from Drosophila melanogaster is presented in SEQ ID NO: 4, and a representative sequence of a human ZIP7 protein is presented in SEQ ID NO: 5. Additional representative sequences are listed in the National Center for Biotechnology Information (NCBI) database. See, for example, NCBI entries: Accession Nos. NM_001077516, NM 006979, NM_001288777, NM_130931, XM_015449564, XM_015449563, XM_005553337, XM_005553336, XM_040983171, XM_040983170, XM_040983169, XM_011976665, XM_011976664, XM_011976663, XM_017522703, XM_017522702, XM_017522700, XM_017522701, XM_021185695, XM_021185696, NM_001048100, XM_038682747, XM_038682746, XM_036766199, XM_036766198, XM_036766197, AQY77122, AQY77121, NP_001070984, NP_008910, and NP_001275706; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce a ZIP7 protein or recombinant polynucleotide comprising a coding sequence encoding a ZIP7 protein for use in the methods described herein.

By “fragment” is intended a molecule consisting of only a part of the intact full-length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full length molecule, but may include at least about 15-25 contiguous amino acid residues of the full length molecule, and can include at least about 20-50 or more contiguous amino acid residues of the full length molecule, or any integer between 5 amino acids and the full length sequence, provided that the fragment in question retains biological activity (e.g., ZIP7 biological activity, ability to enhance ERAD, increase degradation of misfolded proteins, suppress protein aggregation, and/or reduce ER stress).

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “derived from” is used herein to identify the original source of a molecule but is not meant to limit the method by which the molecule is made which can be, for example, by chemical synthesis or recombinant means.

The terms “variant,” “analog” and “mutein” refer to biologically active derivatives of the reference molecule that retain desired activity, such as ZIP7 activity, the ability to improve ERAD function, increase degradation of a misfolded protein, suppress pathological accumulation of misfolded proteins in a cell and/or protein aggregation, and/or reduce ER stress for use in the treatment of a disorder associated with protein misfolding, as described herein. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity, and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same biological activity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, DC, which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as ZIP7 activity, the ability to increase ERAD, increase degradation of a misfolded protein, suppress pathological accumulation of a misfolded protein in a cell and/or protein aggregation, and/or reduce ER stress. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity, and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, adenoviruses, lentiviruses, alphaviruses, pox viruses, and vaccinia viruses.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

Promoting ERAD and Suppressing Accumulation of Misfolded Proteins with ZIP7

The present disclosure provides methods and compositions for enhancing ERAD and suppressing pathological accumulation of misfolded proteins by increasing expression or activity of zinc transporter protein 7 (ZIP7). Methods of treating a subject for a disorder associated with protein misfolding are also provided, including methods of gene therapy for expressing ZIP7 in vivo in effective amounts sufficient to suppress pathological accumulation of misfolded proteins.

A number of neurodegenerative diseases are associated with pathological accumulation of misfolded proteins and protein aggregates in specific regions of the eye, brain, and spinal cord. Without being bound by a particular theory, protein misfolding and aggregation causes ER stress and disrupts endoplasmic reticulum-associated degradation (ERAD). Aggregation of misfolded proteins may also cause cellular dysfunction, loss of synaptic connections, and nerve and brain damage leading to the pathological progression of a neurodegenerative disease. Accumulation of misfolded proteins can be suppressed by promoting ERAD with ZIP7.

Disorders associated with protein misfolding that can be treated with the compositions and methods disclosed herein include any condition resulting in protein misfolding and/or protein aggregation, wherein misfolded proteins and/or protein aggregates accumulate within the endoplasmic reticulum (ER) where they cause ER stress and inhibit ER-associated degradation (ERAD). Disorders associated with protein misfolding include, but are not limited to, retinitis pigmentosa, Huntington's disease, Alzheimer's disease, Parkinson's disease, frontotemporal dementia, amyotrophic lateral sclerosis (ALS), dentatorubropallidoluysian atrophy (DRPLA), spinal and bulbar muscular atrophy (SBMA) also known as Kennedy's disease, multiple system atrophy, and spinocerebellar ataxia (SCA).

### Production of ZIP7

ZIP7 proteins (or biologically active fragments thereof) can be prepared in any suitable manner (e.g., recombinant expression, purification from cell culture, chemical synthesis, etc.) and in various forms (e.g. native, fusions, labeled, lipidated, amidated, acetylated, PEGylated, etc.). The ZIP7 proteins may include naturally occurring polypeptides, recombinantly produced polypeptides, synthetically produced polypeptides, or polypeptides produced by a combination of these methods. Means for preparing proteins are well understood in the art. Proteins are preferably prepared in substantially pure form (i.e. substantially free from other host cell or non-host cell proteins).

ZIP7 nucleic acid and protein sequences may be derived from any source. A number of ZIP7 nucleic acid and protein sequences are known. Representative ZIP7 sequences are presented in SEQ ID NO:4 for ZIP7 from Drosophila melanogaster and SEQ ID NO:5 for ZIP7 from Homo sapiens, and additional representative sequences are listed in the National Center for Biotechnology Information (NCBI) database. See, for example, NCBI entries: Accession Nos. NM_001077516, NM 006979, NM_001288777, NM_130931, XM_015449564, XM_015449563, XM_005553337, XM_005553336, XM_040983171, XM_040983170, XM_040983169, XM_011976665, XM_011976664, XM_011976663, XM_017522703, XM_017522702, XM_017522700, XM_017522701, XM_021185695, XM_021185696, NM 001048100, XM_038682747, XM_038682746, XM_036766199, XM_036766198, XM_036766197, AQY77122, AQY77121, NP_001070984, NP_008910, and NP_001275706; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce a ZIP7 protein or a recombinant polynucleotide comprising a coding sequence encoding a ZIP7 protein for use in the methods described herein. In certain embodiments, the ZIP7 protein used to promote ERAD comprises or consists of the amino acid sequence of SEQ ID NO:5, or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, or a biologically active fragment thereof, wherein the ZIP7 is capable of increasing ERAD in a cell and suppressing pathological accumulation of misfolded proteins.

In one embodiment, ZIP7 proteins are generated using recombinant techniques. One of skill in the art can readily determine nucleotide sequences that encode the desired protein using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding proteins can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding proteins that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified proteins can be generated by creating expression constructs which operably link, in various combinations, polynucleotides encoding proteins having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage k (E. coli), pBR322 (E. co/i), pACYC177 (E. co/i), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA (“MaxBac” kit).

Plant expression systems can also be used to produce the ZIP7 proteins. Generally, such systems use virus-based vectors to transfect plant cells with heterologous genes. For a description of such systems, see, e.g., Porta et al., Mol. Biotech. (1996) 5:209-221; and Hackland et al., Arch. Virol. (1994) 139:1-22.

Viral systems, such as a vaccinia-based infection/transfection system, as described in Tomei et al., J Virol. (1993) 67:4017-4026 and Selby et al., J Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honeybee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases, it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fusion proteins of the present invention are produced by growing host cells transformed by an expression vector described above under conditions whereby the protein of interest is expressed. The selection of the appropriate growth conditions is within the skill of the art.

In one embodiment, the transformed cells secrete the ZIP7 protein product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted ZIP7 protein product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant peptides or polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced peptides or polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular peptides or polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the peptides or polypeptides can be further purified using conventional techniques well known in the art, such as by any of the techniques described above.

The ZIP7 proteins can be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1st ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final peptide or polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, IL 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The ZIP7 proteins can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

### Pharmaceutical Compositions

A ZIP7 protein or a recombinant polynucleotide comprising a coding sequence encoding a ZIP7 protein can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the ZIP7 proteins, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the ZIP7 protein (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising a ZIP7 protein are in unit dosage form, meaning an amount of a conjugate or composition appropriate for a single dose, in a premeasured or pre-packaged form.

The compositions herein may optionally include one or more additional agents, such as other drugs for treating a disorder associated with protein misfolding, or other medications used to treat a subject for a condition or disease. Compounded preparations may include a ZIP7 protein and one or more drugs for treating a disorder associated with protein misfolding, such as tetrabenazine, amantadine, neuroleptics (e.g., butyrophenones, diphenylbutylpiperidines, phenothiazines, thioxanthenes, benzamides, tricyclics, and benzisoxazoles/benzisothiazoles), benzodiazepines (e.g., alprazolam, flunitrazepam, chlordiazepoxide, clonazepam, diazepam, lorazepam, midazolam, oxazepam, and prazepam), cholinesterase inhibitors (e.g., Razadyne (galantamine), Exelon (rivastigmine), Aricept (donepezil), and Cognex (tacrine)), N-methyl D-aspartate (NMDA) antagonists (e.g., Namenda (memantine), remacemide), selective serotonin reuptake inhibitors (e.g., citalopram, escitalopram, fluoxetine, fluvoxamine, paroxetine, and sertraline), anticonvulsants (e.g., paraldehyde, stiripentol, barbiturates such as phenobarbital, methylphenobarbital, and barbexaclone, carboxamides such as carbamazepine, oxcarbazepine, and eslicarbazepine acetate, fatty acids such as valproates, vigabatrin, progabide, and tiagabine, fructose derivatives such as topiramate, gamma-aminobutyric acid (GABA) analogs such as gabapentin, pregabalin, vigabatrin, and progabide, and hydantoins such as ethotoin, phenytoin, mephenytoin, and fosphenytoin), vitamin A, docosahexaenoic acid (DHA), and lutein, or other medications. Alternatively, such agents can be contained in a separate composition from the composition comprising a ZIP7 protein and co-administered concurrently, before, or after the composition comprising the ZIP7 protein.

### Nucleic Acids Encoding ZIP7

Nucleic acids encoding ZIP7 can be used to treat a disorder associated with protein misfolding. Nucleic acids described herein can be inserted into an expression vector to create an expression cassette capable of producing the ZIP7 in a suitable host cell. The ability of constructs to produce the ZIP7 can be empirically determined.

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding ZIP7 can be administered to a subject using standard gene delivery protocols. Methods for gene delivery are known in the art. See, e.g., U.S. Pat. Nos. 5,399,346, 5,580,859, 5,589,466. Genes can be delivered either directly to a subject or, alternatively, delivered ex vivo, to cells derived from the subject and the cells reimplanted in the subject.

A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides encoding ZIP7 is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the ZIP7 include those derived from the pox family of viruses, including vaccinia virus and avian poxvirus. By way of example, vaccinia virus recombinants expressing the ZIP7 can be constructed as follows. The DNA encoding the particular ZIP7 coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the coding sequences of interest (for example, a ZIP7 expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, AL), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in Methods of Immunology (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering synthetic expression cassettes encoding ZIP7. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors carrying a synthetic expression cassette encoding ZIP7 are formulated into compositions for delivery to a vertebrate subject. These compositions may either be prophylactic (to prevent accumulation and/or aggregation of a misfolded protein) or therapeutic (to treat a disorder associated with protein misfolding). The compositions will comprise a “therapeutically effective amount” of the nucleic acid of interest such that an amount of the ZIP7 protein (or a biologically active fragment thereof) can be produced in vivo so that ERAD is increased and accumulation of misfolded proteins is decreased in the individual to which it is administered. The exact amount necessary will vary depending on the subject being treated; the age and general condition of the subject to be treated; the degree of protection desired; the severity of the condition being treated; the particular ZIP7 protein produced and its mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject™ or a gene gun, such as the Accell™ gene delivery system (PowderMed Ltd, Oxford, England).

### Administration

At least one therapeutically effective cycle of treatment with a ZIP7 protein or a recombinant polynucleotide comprising a coding sequence encoding ZIP7 will be administered to a subject for treatment of a disorder associated with protein misfolding. Disorders associated with protein misfolding include, but are not limited to, retinitis pigmentosa, Huntington's disease, Alzheimer's disease, Parkinson's disease, frontotemporal dementia, amyotrophic lateral sclerosis (ALS), dentatorubropallidoluysian atrophy (DRPLA), spinal and bulbar muscular atrophy (SBMA) also known as Kennedy's disease, multiple system atrophy, and spinocerebellar ataxia (SCA).

By “therapeutically effective dose or amount” of a ZIP7 protein or a recombinant polynucleotide comprising a coding sequence encoding a ZIP7 protein is intended an amount that, when administered, as described herein, brings about a positive therapeutic response, such as improved recovery from a disorder associated with protein misfolding. Improved recovery may include improved ERAD function resulting in increased degradation of the misfolded protein and reduced formation of protein aggregates, restored neuronal function, improved cognition, improved memory, or increased survival. In the case of retinitis pigmentosa, a therapeutically effective dose or amount of a ZIP7 protein or a recombinant polynucleotide comprising a coding sequence encoding a ZIP7 protein may reduce accumulation of misfolded rhodopsin and rhodopsin protein aggregates in the ER and photoreceptors in the retina of the eye, reduce ER stress, improve survival of photoreceptor cells, and prevent or delay eye damage and loss of vision. In the case of an amyloid beta aggregation-associated disease (e.g., Alzheimer's disease), a therapeutically effective dose or amount” of a ZIP7 protein may reduce amyloid beta aggregation and reduce formation of amyloid plaques in the brain. Additionally, a therapeutically effective dose or amount may retard loss of cerebellar Purkinje neurons and loss of brain cells. In the case of an alpha-synuclein aggregation-associated disease or synucleinopathy (e.g., Parkinson's disease), a therapeutically effective dose or amount” of a ZIP protein may reduce aggregation of alpha-synuclein. Additionally, a therapeutically effective dose or amount may reduce accumulation of aggregates of alpha-synuclein in neurons, nerve fibers, and/or glial cells and reduce formation of Lewy bodies.

In certain embodiments, multiple therapeutically effective doses of compositions comprising a ZIP7 protein, or a recombinant polynucleotide comprising a coding sequence encoding ZIP7, and/or one or more other therapeutic agents, such as other drugs for treating a disorder associated with protein misfolding, or other medications will be administered. The compositions of the present invention are typically, although not necessarily, administered orally, via injection (subcutaneously, intravenously, or intramuscularly), by infusion, or locally. Additional modes of administration are also contemplated, such as intracerebroventricular, intracerebral, intraneural, intraspinal, intralesion, intraparenchymatous, pulmonary, rectal, transdermal, transmucosal, intrathecal, pericardial, intra-arterial, intraocular, intraperitoneal, and so forth. In particular embodiments, compositions are administered into the eye, brain, spinal cord, or cerebrospinal fluid of a subject.

The preparations are also suitable for local treatment. In a particular embodiment, a composition is used for localized delivery of a ZIP7 protein or a recombinant polynucleotide comprising a coding sequence encoding ZIP7 for the treatment of a disorder associated with protein misfolding. For example, compositions may be administered directly into a photoreceptor or neuron or by stereotactic injection into the brain. The particular preparation and appropriate method of administration are chosen to target the ZIP7 protein or recombinant polynucleotide comprising a coding sequence encoding the ZIP7 protein to the site of aberrant accumulation of a misfolded protein or protein aggregation (e.g., misfolded, dysfunctional rhodopsin in photoreceptors, insoluble polyQ protein aggregates in the nuclei of neurons, or amyloid beta plaques in the brain).

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising a ZIP7 protein and/or other agents may be administered using the same or different routes of administration in accordance with any medically acceptable method known in the art.

In another embodiment, the pharmaceutical compositions comprising a ZIP7 protein or a recombinant polynucleotide comprising a coding sequence encoding a ZIP7 protein, and/or other agents are administered prophylactically, e.g., to prevent accumulation of misfolded proteins and/or protein aggregation (e.g., rhodopsin, polyQ, or amyloid beta accumulation or aggregation). Such prophylactic uses will be of particular value for subjects who have a genetic predisposition to developing a disorder associated with protein misfolding. In another embodiment, the pharmaceutical compositions comprising a ZIP7 protein or a recombinant polynucleotide comprising a coding sequence encoding a ZIP7 protein and/or other agents are administered therapeutically to subjects with symptoms such as loss of vision, dementia, loss of mental acuity, or loss of muscle coordination caused by a disorder associated with protein misfolding.

In another embodiment, the pharmaceutical compositions comprising a ZIP7 protein or a recombinant polynucleotide comprising a coding sequence encoding a ZIP7 protein and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The disclosure also provides a method for administering a conjugate comprising a ZIP7 protein to a patient suffering from a disorder associated with protein misfolding or condition that is responsive to treatment with a ZIP7 protein contained in the conjugate or composition. The method comprises administering, via any of the herein described modes, a therapeutically effective amount of the conjugate or drug delivery system, preferably provided as part of a pharmaceutical composition. The method of administering may be used to treat any condition that is responsive to treatment with a ZIP7 protein.

Those of ordinary skill in the art will appreciate which conditions a specific a ZIP7 protein can effectively treat. The actual dose to be administered will vary depending upon the age, weight, and general condition of the subject as well as the particular disorder associated with protein misfolding being treated, the severity of the condition being treated, the judgment of the health care professional, and the particular ZIP7 protein or conjugate being administered. Therapeutically effective amounts can be determined by those skilled in the art, and will be adjusted to the particular requirements of each particular case.

In certain embodiments, multiple therapeutically effective doses of a ZIP7 protein will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising a ZIP7 protein will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present disclosure, this type of dosing is referred to as “intermittent” therapy. In accordance with the methods described herein, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the specific ZIP7 protein, the particular disorder associated with protein misfolding that is treated, the magnitude of the effect desired, and the route of administration.

A purified ZIP7 protein or a recombinant polynucleotide comprising a coding sequence encoding a ZIP7 protein (again, preferably provided as part of a pharmaceutical preparation) can be administered alone or in combination with one or more other therapeutic agents, such as tetrabenazine, amantadine, neuroleptics (e.g., butyrophenones, diphenylbutylpiperidines, phenothiazines, thioxanthenes, benzamides, tricyclics, and benzisoxazoles/benzisothiazoles), benzodiazepines (e.g., alprazolam, flunitrazepam, chlordiazepoxide, clonazepam, diazepam, lorazepam, midazolam, oxazepam, and prazepam), cholinesterase inhibitors (e.g., Razadyne (galantamine), Exelon (rivastigmine), Aricept (donepezil), and Cognex (tacrine)), N-methyl D-aspartate (NMDA) antagonists (e.g., Namenda (memantine), remacemide), selective serotonin reuptake inhibitors (e.g., citalopram, escitalopram, fluoxetine, fluvoxamine, paroxetine, and sertraline), anticonvulsants (e.g., paraldehyde, stiripentol, barbiturates such as phenobarbital, methylphenobarbital, and barbexaclone, carboxamides such as carbamazepine, oxcarbazepine, and eslicarbazepine acetate, fatty acids such as valproates, vigabatrin, progabide, and tiagabine, fructose derivatives such as topiramate, gamma-aminobutyric acid (GABA) analogs such as gabapentin, pregabalin, vigabatrin, and progabide, and hydantoins such as ethotoin, phenytoin, mephenytoin, and fosphenytoin), vitamin A, docosahexaenoic acid (DHA), and lutein, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A ZIP7 protein or a recombinant polynucleotide comprising a coding sequence encoding a ZIP7 protein can be administered prior to, concurrent with, or subsequent to other agents. If provided at the same time as other agents, the ZIP7 protein or recombinant polynucleotide comprising a coding sequence encoding the ZIP7 protein can be provided in the same or in a different composition. Thus, ZIP7 protein or recombinant polynucleotide comprising a coding sequence encoding the ZIP7 protein and/or other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a ZIP7 protein or a recombinant polynucleotide comprising a coding sequence encoding a ZIP7 protein, and a dose of a pharmaceutical composition comprising at least one other agent, such as another drug for treating a disorder associated with protein misfolding, which in combination comprise a therapeutically effective dose, according to a particular dosing regimen. Similarly, a ZIP7 protein or a recombinant polynucleotide comprising a coding sequence encoding a ZIP7 protein and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

### Kits

Also provided are kits for treating a patient for a disorder associated with protein misfolding with a ZIP7 protein or a recombinant polynucleotide comprising a coding sequence encoding a ZIP7 protein, as described herein. The ZIP7 protein or recombinant polynucleotide comprising a coding sequence encoding the ZIP7 protein and optionally other therapeutic agents may be contained in separate compositions or in the same composition. Kits may include unit doses of the formulations comprising the ZIP7 protein suitable for use in the treatment methods described herein, e.g., in tablets or injectable dose(s). In such kits, in addition to the containers containing the unit doses will be an informational package insert describing the use and attendant benefits of the treatment for a disorder associated with protein misfolding. The kit can include, for example, a dosing regimen for the ZIP7 protein.

Formulations suitable for intravenous administration are of particular interest, and in such embodiments the kit may further include a syringe or other device to accomplish such administration, which syringe or device may be pre-filled with the ZIP7 protein. The instructions can be printed on a label affixed to the container or can be a package insert that accompanies the container.

In certain embodiments, the kit comprises a ZIP7 protein comprising or consisting of the amino acid sequence of SEQ ID NO:5, or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, or a biologically active fragment thereof, wherein the ZIP7 is capable of increasing ERAD in a cell and suppressing pathological accumulation of misfolded proteins. A subject kit may include at least one container comprising a solution comprising a unit dose of the ZIP7 protein, and a pharmaceutically acceptable excipient; and instructions to administer a unit dose according to a desired regimen or exemplary regimen dependent upon the particular disorder associated with protein misfolding being treated, age, weight, and the like.

### Utility

The compositions and methods of the present disclosure find use in a variety of different applications, including the treatment of conditions resulting in protein misfolding and/or protein aggregation (i.e., disorders associated with protein misfolding), wherein misfolded proteins and/or protein aggregates accumulate within the endoplasmic reticulum (ER), cause ER stress, and inhibit ER-associated degradation (ERAD). Disorders associated with protein misfolding include, but are not limited to, retinitis pigmentosa and neurodegenerative diseases such as Huntington's disease, Alzheimer's disease, Parkinson's disease, frontotemporal dementia, amyotrophic lateral sclerosis (ALS), dentatorubropallidoluysian atrophy (DRPLA), spinal and bulbar muscular atrophy (SBMA) also known as Kennedy's disease, multiple system atrophy, and spinocerebellar ataxia (SCA). Aggregation of misfolded proteins may cause cellular dysfunction, loss of synaptic connections, neuron death, and/or brain damage. Treatment with ZIP7, or a recombinant polynucleotide comprising a coding sequence encoding ZIP7, may reduce or prevent accumulation or aggregation of misfolded proteins, improve cellular function, and delay or prevent loss of synaptic connections, neuron death, loss of vision, or brain damage.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of providing a subject with zinc transporter protein 7 (ZIP7) to suppress pathological accumulation of a misfolded protein, the method comprising introducing an expression vector comprising a promoter operably linked to a coding sequence encoding the ZIP7 into a cell, wherein the cell expresses the ZIP in vivo in the subject in an effective amount sufficient to suppress the pathological accumulation of the misfolded protein in the cell.
2. The method of aspect 1, wherein the cell is a retina cell or a brain cell.
3. The method of aspect 2, wherein the retina cell is a photoreceptor cell.
4. The method of aspect 2 or 3, wherein the misfolded protein is a misfolded rhodopsin protein.
5. The method of any one of aspects 1-4, wherein the expression vector is introduced into the cell ex vivo or in vivo.
6. The method of any one of aspects 1-5, wherein the subject has a disorder associated with protein misfolding.
7. The method of any one of aspect 6, wherein the disorder associated with protein misfolding is retinitis pigmentosa.
8. The method of aspect 6, wherein the disorder associated with protein misfolding is a neurodegenerative disease.
9. The method of any one of aspects 1-8, wherein the misfolded protein is Rh1G69D rhodopsin, Vap33, or amyloid β42.
10. A method of treating a subject for a disorder associated with protein misfolding, the method comprising administering an expression vector comprising a promoter operably linked to a nucleotide sequence encoding zinc transporter protein 7 (ZIP7) to the subject, wherein the ZIP7 is expressed in vivo in the subject in a therapeutically effective amount sufficient to suppress pathological accumulation of the misfolded protein.
11. The method of aspect 10, wherein the disorder associated with protein misfolding is retinitis pigmentosa or a neurodegenerative disease.
12. The method of aspect 10 or 11, wherein the expression vector is administered locally into an eye or the brain of the subject.
13. The method of aspect 10 or 11, wherein the expression vector is administered locally into a retina or a photoreceptor of the subject.
14. The method of any one of aspects 10-13, wherein the misfolded protein is Rh1G69D rhodopsin, Vap33, or amyloid β42.
15. A method of treating a subject for a disorder associated with protein misfolding, the method comprising administering to the subject a therapeutically effective amount of a zinc transporter protein 7 (ZIP7) protein.
16. The method of aspect 15, wherein the ZIP7 protein comprises or consists of an amino acid sequence having at least 90% identity to the sequence of SEQ ID NO: 5.
17. The method of aspect 15 or 16, wherein the disorder associated with protein misfolding is retinitis pigmentosa or a neurodegenerative disease.
18. The method of any one of aspects 15-17, wherein the ZIP7 protein is administered locally into an eye or the brain of the subject.
19. The method of any one of aspects 15-18, wherein the ZIP7 protein is administered locally into a retina or a photoreceptor of the subject.
20. The method of any one of aspects 15-19, wherein the ZIP7 protein is administered according to a daily dosing regimen or intermittently.
21. The method of any one of aspects 15-20, wherein the misfolded protein is Rh1G69D rhodopsin, Vap33, or amyloid β42.
22. A method of enhancing endoplasmic reticulum (ER)-associated degradation (ERAD) or proteosome-associated degradation in a cell, the method comprising increasing expression or activity of zinc transporter protein 7 (ZIP7) in the cell.
23. The method of aspect 22, wherein the cell is a retina cell.
24. The method of aspect 23, wherein the retina cell is a photoreceptor cell.
25. The method of aspect 23 or 24, wherein said increasing expression or activity of ZIP7 is sufficient to increase degradation of a misfolded rhodopsin protein and suppress accumulation of the misfolded rhodopsin protein in the retina cell.
26. The method of any one of aspects 22-25, wherein said increasing expression of ZIP7 comprises transfecting the cell with a recombinant polynucleotide comprising a coding sequence encoding ZIP7.
27. The method of aspect 26, wherein the recombinant polynucleotide is provided by a viral vector comprising a promoter operably linked to the coding sequence encoding the ZIP7.
28. The method of aspect 27, wherein the coding sequence encoding the ZIP7 is integrated into a chromosomal locus of the transfected cell.
29. The method of aspect 28, wherein an endogenous promoter is operably linked to the integrated coding sequence encoding the ZIP7 at the chromosomal locus.
30. A method of suppressing accumulation of a misfolded protein in an organ, cell, or tissue of a subject, the method comprising increasing expression or activity of zinc transporter protein 7 (ZIP7) in the organ, cell, or tissue.
31. The method of aspect 30, wherein the organ is an eye or a brain.
32. The method of aspect 30, wherein the tissue is neural tissue.
33. The method of aspect 30, wherein the tissue is retina tissue.
34. The method of aspect 30, wherein the cell is a retina cell.
35. The method of aspect 34, wherein the retina cell is a photoreceptor cell.
36. The method of aspect 35, wherein the misfolded protein is a rhodopsin protein.
37. The method of any one of aspects 30-36, wherein the subject has retinitis pigmentosa.
38. The method of any one of aspects 30-37, wherein the subject has a disorder associated with protein misfolding.
39. The method of aspect 38, wherein the disorder associated with protein misfolding is a neurodegenerative disease.
40. The method of any one of aspects 30-39, wherein the expression of ZIP7 is increased sufficiently to decrease pathological accumulation of a misfolded protein and increase cell survival.
41. The method of any one of aspects 30-40, wherein said increasing expression of ZIP7 comprises providing a recombinant polynucleotide comprising a coding sequence encoding the ZIP7 to the organ, cell, or tissue, wherein the ZIP7 is expressed in the organ, cell, or tissue.
42. The method of aspect 41, wherein the recombinant polynucleotide further comprises a viral vector comprising a promoter operably linked to the coding sequence encoding the ZIP7.
43. The method of aspect 42, wherein the coding sequence encoding the ZIP7 is integrated into a chromosomal locus.
44. The method of aspect 43, wherein an endogenous promoter is operably linked to the integrated coding sequence encoding the ZIP7 at the chromosomal locus.
45. The method of any one of aspects 30-44, wherein the misfolded protein is Rh1G69D rhodopsin, Vap33, or amyloid β42.
46. A composition for use in a method of treating a disorder associated with protein misfolding, the composition comprising zinc transporter protein 7 (ZIP7) or an expression vector comprising a promoter operably linked to a coding sequence encoding ZIP7.
47. The composition of aspect 46, further comprising a pharmaceutically acceptable excipient.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for. Those of skill in the art will readily recognize a variety of noncritical parameters that could be changed or modified to yield essentially similar results.

### Example 1: Collective Border Cell Migration Requires the Zn²⁺ Transporter Catsup to Promote Endoplasmic Reticulum-Associated Protein Degradation

Here we study Catsup function in border cells. Together with published results, our data suggest a unified model for Catsup/ZIP7 in providing rate-limiting Zn² for degradation of ER-localized misfolded proteins, thereby alleviating ER stress to promote cell survival, migration, and Notch transcriptional responses.

### Results

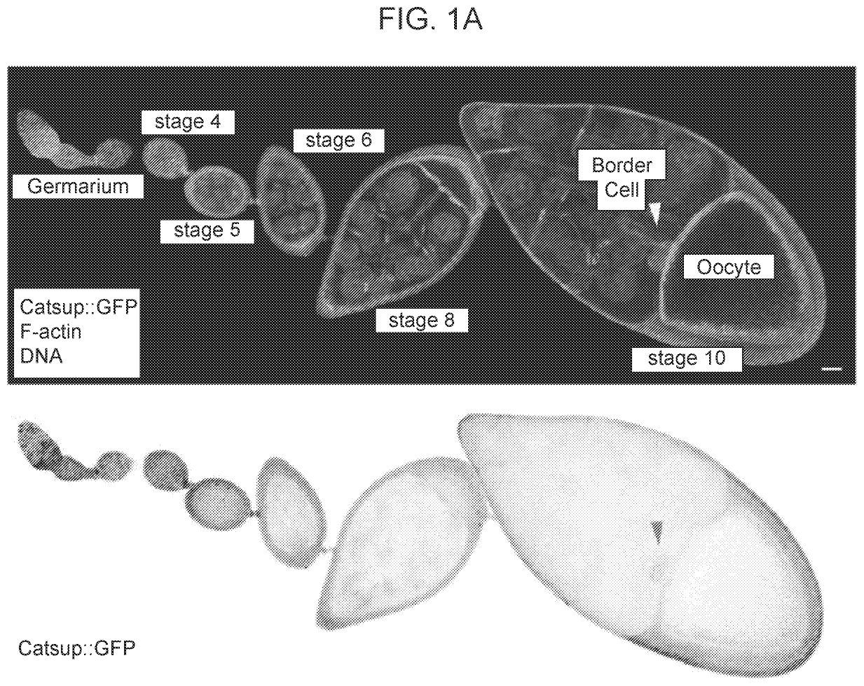

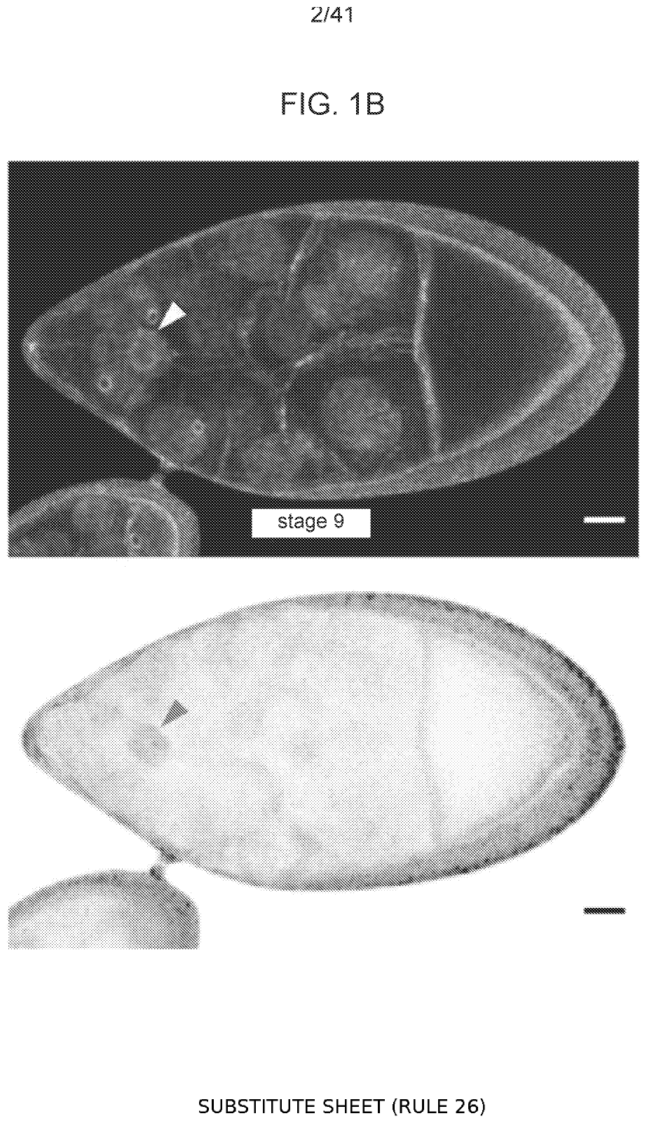

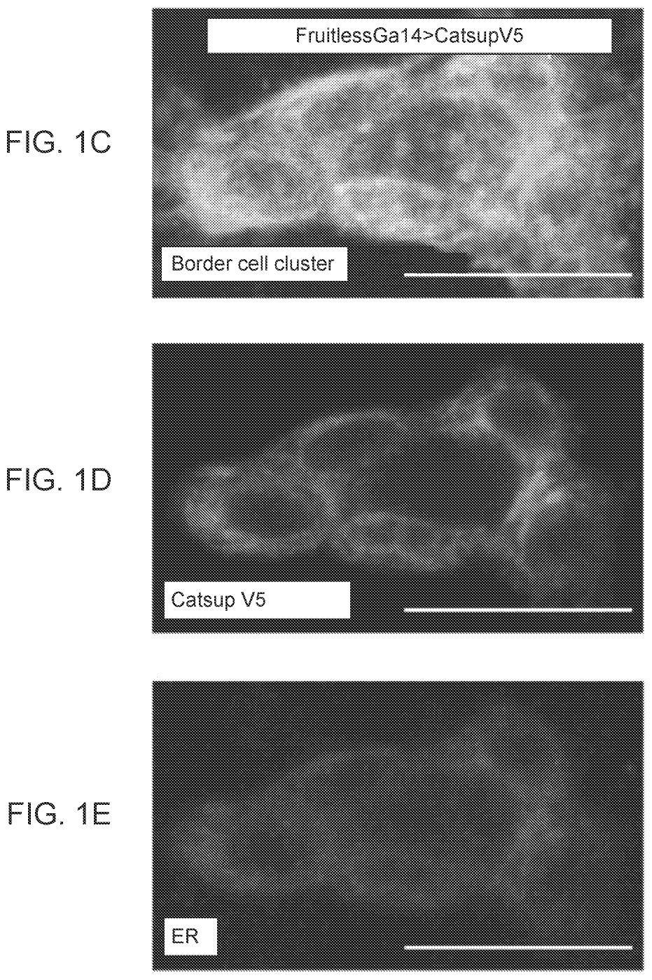

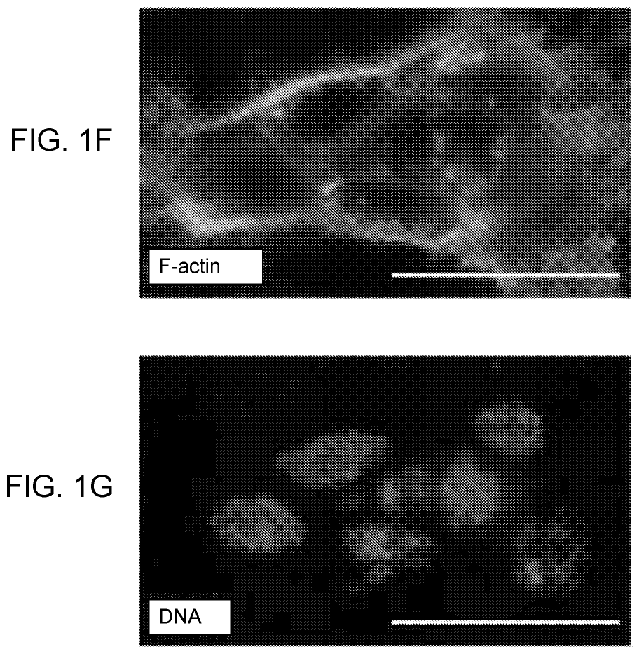

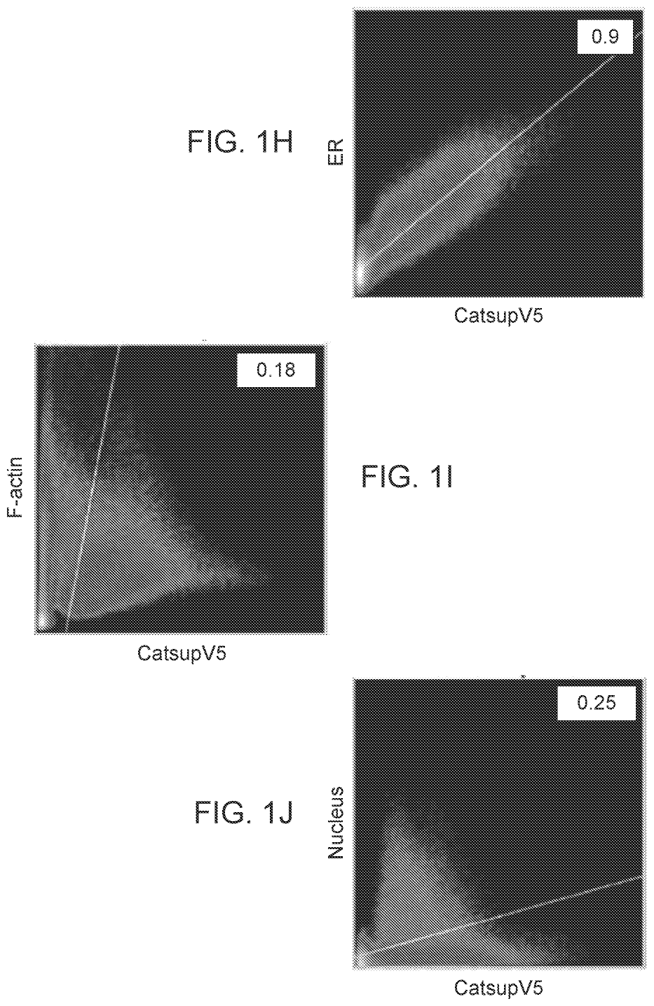

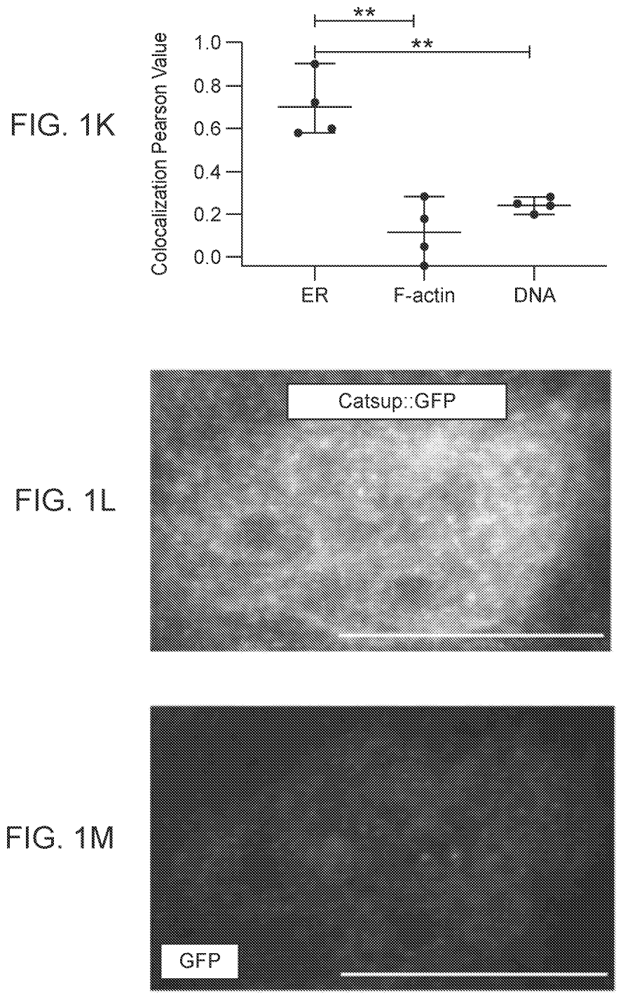

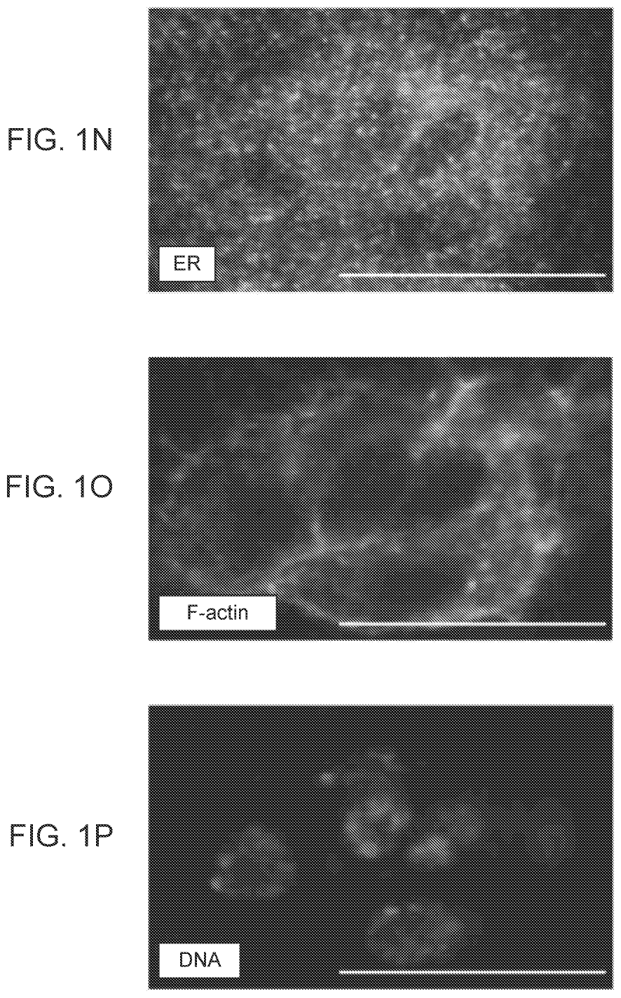

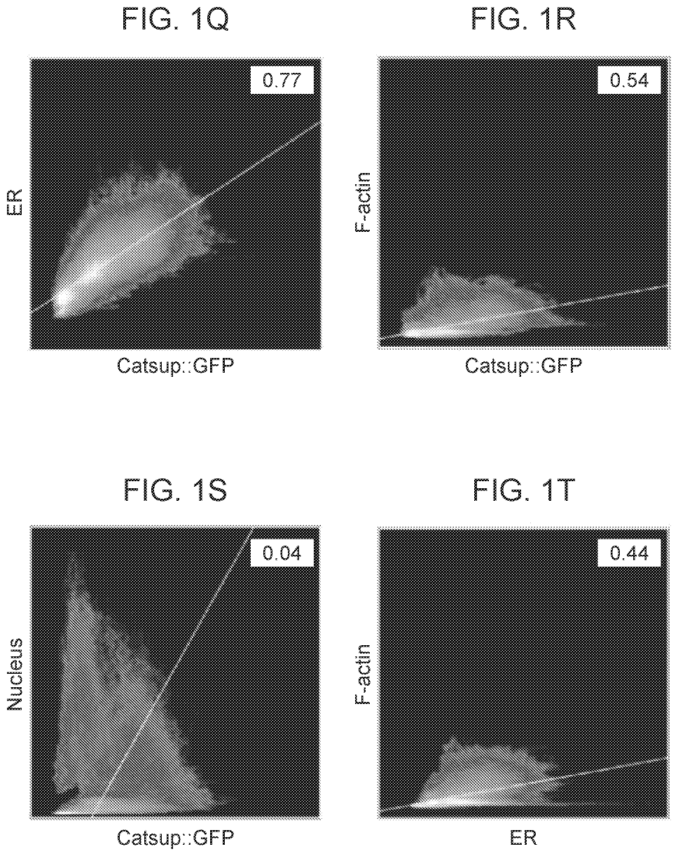

To study the roles of Catsup in the ovary, we first examined its expression using a Catsup::GFP fusion protein expressed under endogenous genomic regulatory sequences⁵⁰. Catsup::GFP was expressed throughout oogenesis, including in all follicle cells (FIGS. 1A-1B). Mammalian ZIP7 localizes predominantly to the ER⁴², and both over-expressed CatsupV5 (FIGS. 1C-1K) and Catsup::GFP (FIGS. 1L-1T) significantly co-localized with the ER resident protein-folding enzyme, Protein Disulfide Isomerase (PDI), but not with DNA or F-actin, consistent with earlier findings in wing imaginal discs⁴⁰.

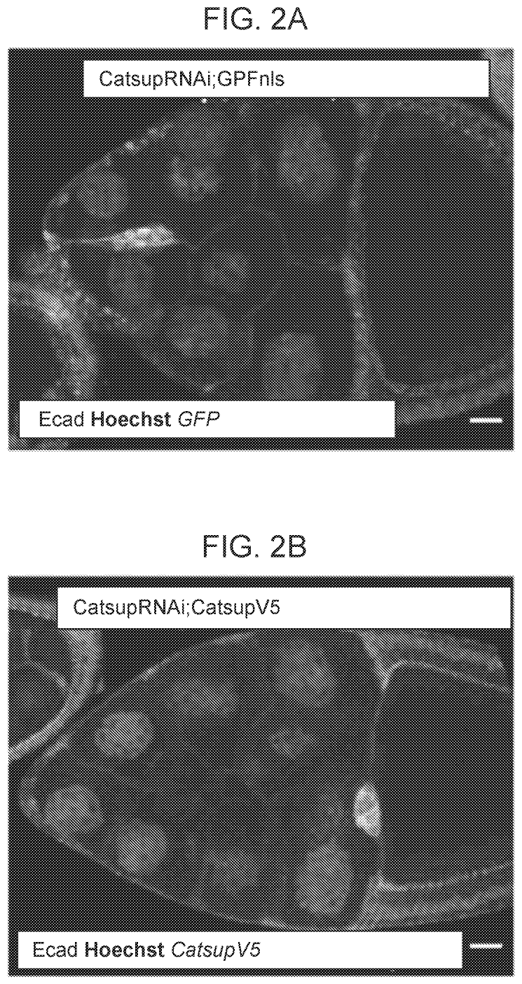

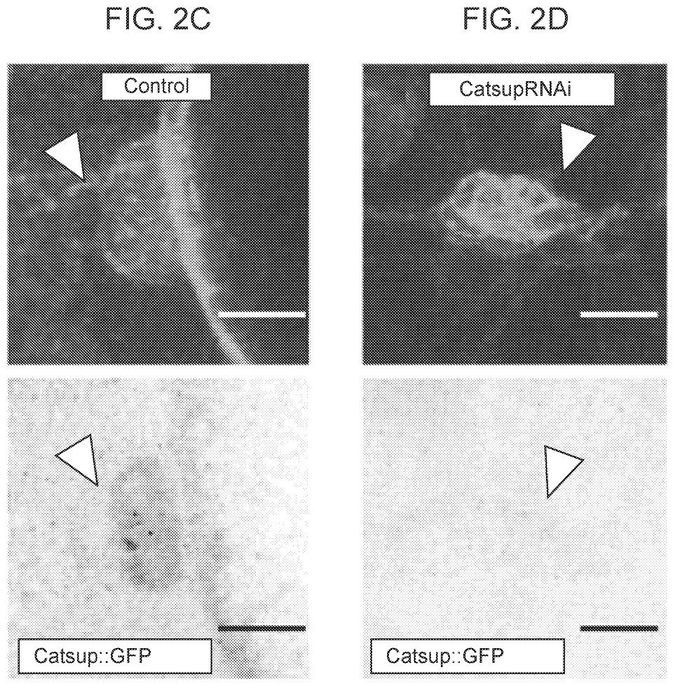

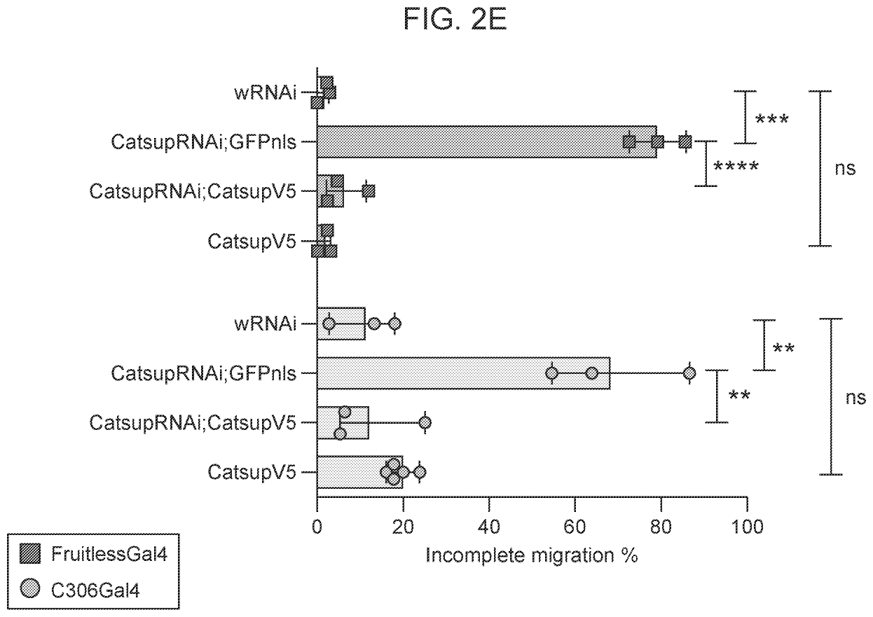

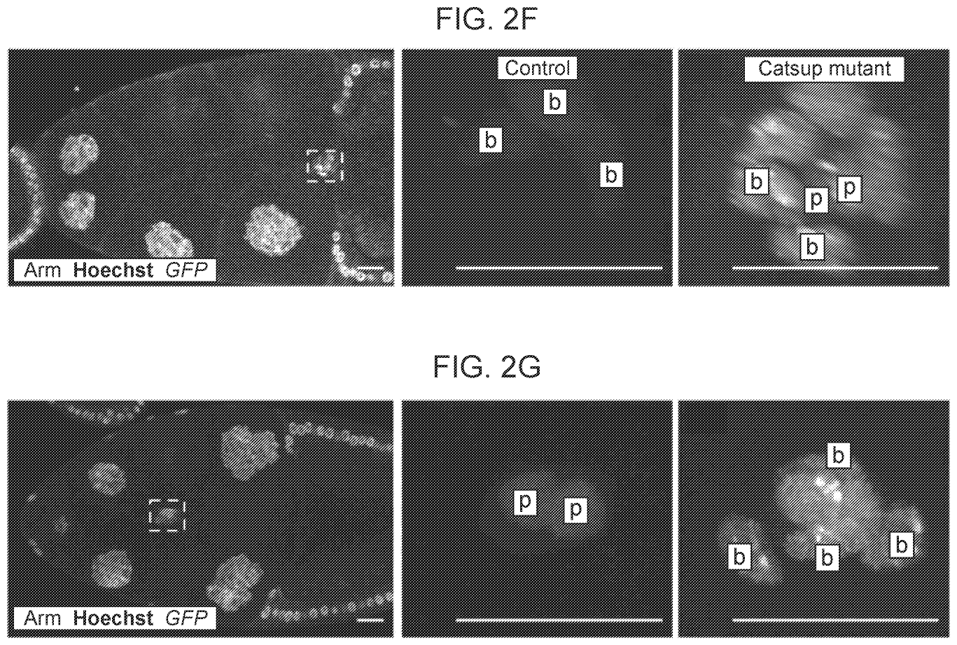

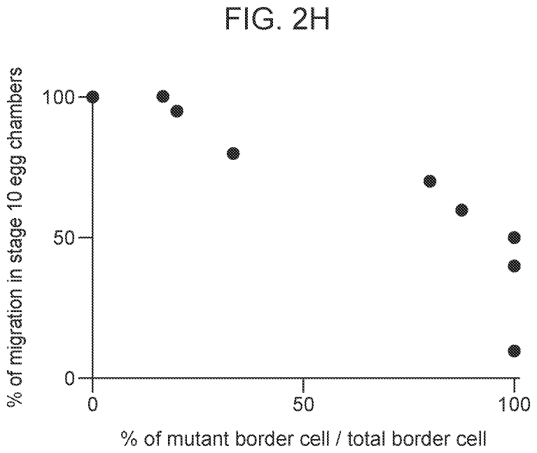

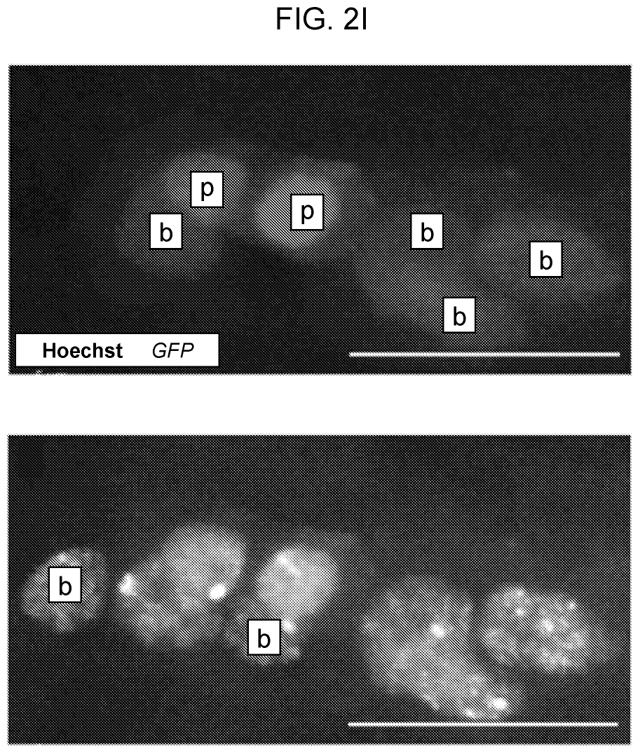

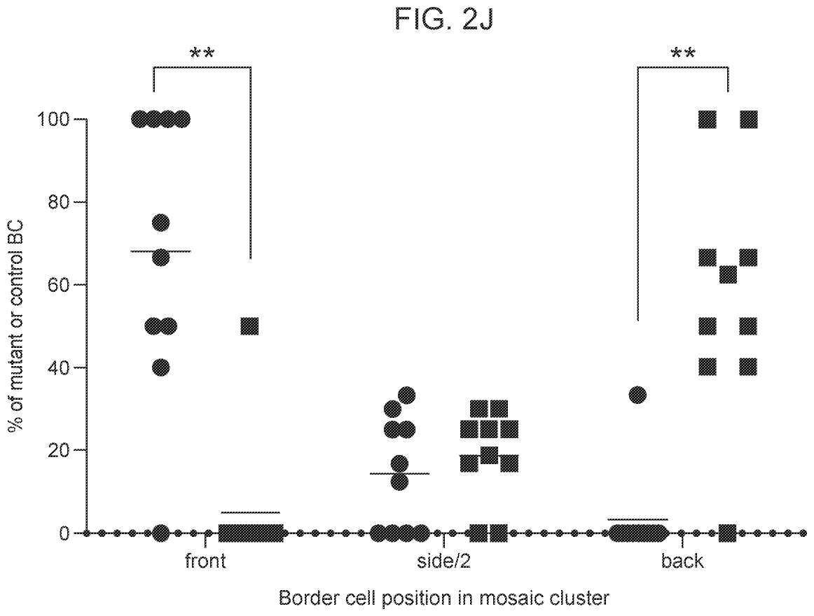

Border cell clusters are composed of 4-6 migratory cells that surround and carry two non-migratory polar cells. Expression of a Catsup RNAi line in outer, migratory border cells using fruitlessGal4⁵¹ inhibited migration (FIG. 2A). The defect was rescued by co-expression of UAS-CatsupV5 (FIG. 2B). Reduction of Catsup::GFP confirmed the effectiveness of the RNAi (FIGS. 2C, C′ and 2D, D′). Border cell migration was also impaired when Catsup RNAi was driven by c306Gal4 (FIG. 2E), which is expressed in both polar and migratory cells. FruitlessGal4-driven RNAi impaired border cell migration at least as much as c306Gal4, indicating that Catsup was primarily required in the outer, migratory cells (FIG. 2E).

As a further test of cell autonomy, we used the FLP-FRT system to generate mosaic egg chambers with clones of homozygous Catsup mutant cells. No migration was detected in homozygous mutant polar cells (FIG. 2F). However, when border cell clusters contained homozygous mutant outer border cells, migration was impaired. Furthermore, the magnitude of the defect correlated with the proportion of mutant cells (FIGS. 2G-2H). In clusters containing both heterozygous and homozygous mutant cells, homozygous mutant cells tended to occupy rear positions (FIGS. 2I-2J), which is typical of mutations in genes required autonomously for motility⁵². We conclude that Catsup is required in outer migratory border cells for motility.

Catsup mutant wing disc epithelial cells are prone to apoptosis⁴⁰, and we observed that 33% (112/337) of Catsup mutant follicular epithelial cells were positive for cleaved and activated caspase, which is indicative of cells undergoing apoptosis. However, we never detected cleaved caspase in Catsup mutant border cells (0/32), indicating that border cells are more resistant to death, consistent with our earlier report that border cells mutant for thread, which encodes the Drosophila inhibitor of apoptosis protein (DIAP1), are viable⁵³.

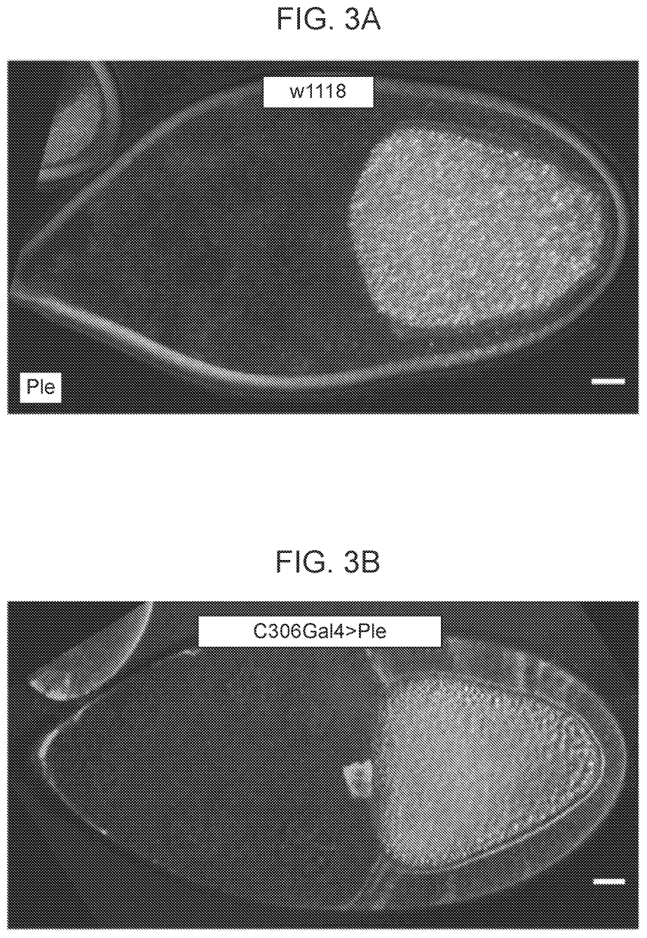

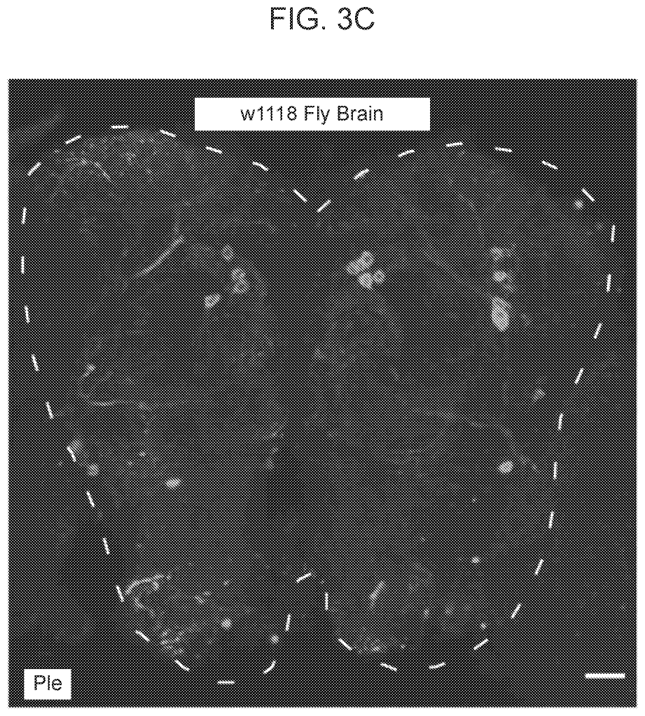

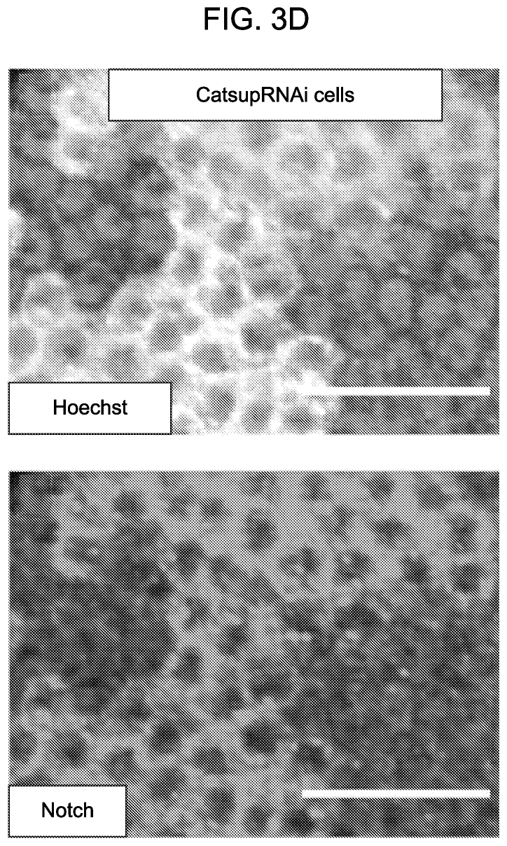

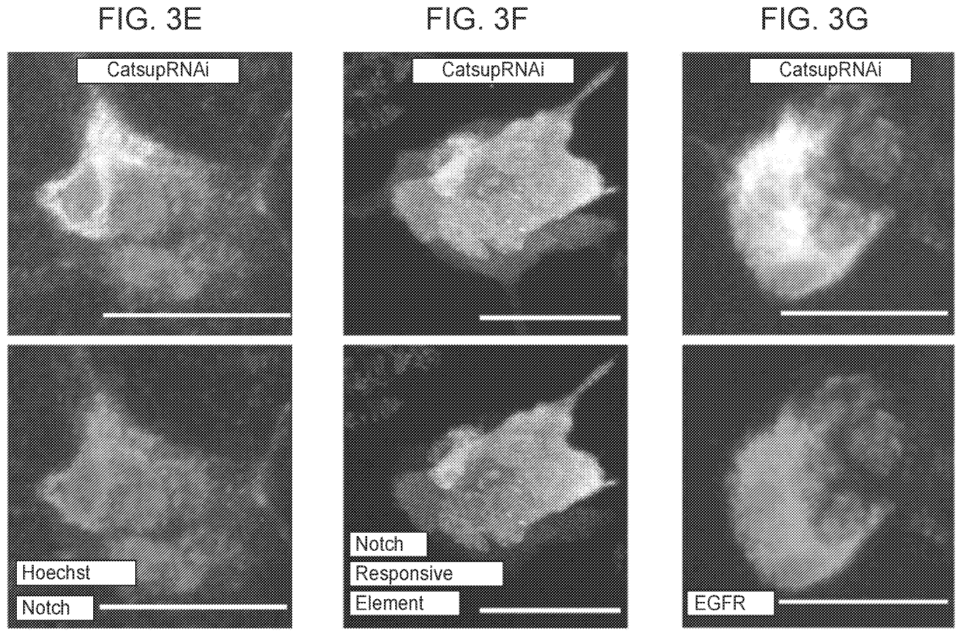

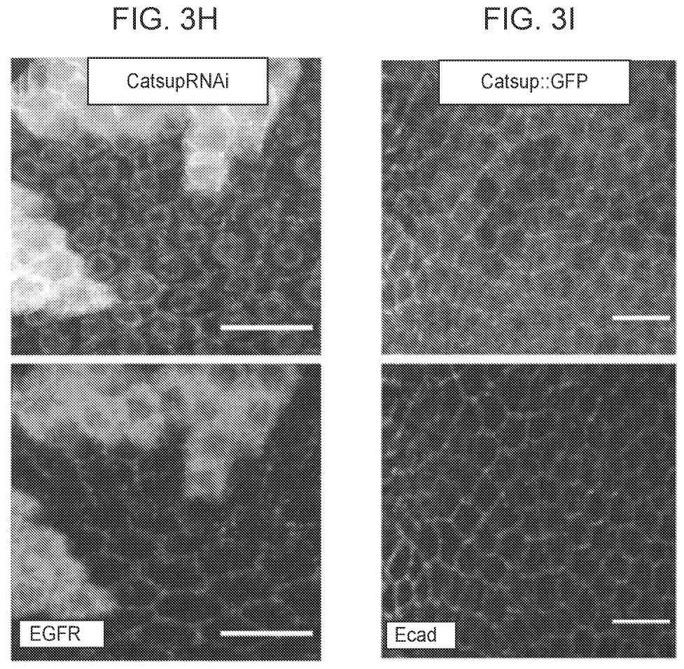

One known function of Catsup is direct binding and inhibition of the tyrosine hydroxylase Ple, which is the rate-limiting enzyme in catecholamine synthesis⁵⁴. Ple and Catsup are expressed in embryonic tracheal cells, where they contribute to achieving proper dopamine levels, which regulate Breathless (fibroblast growth factor receptor) endocytosis and signaling³⁹. To test whether Catsup functions similarly in border cell migration, we used an antibody to assess Ple expression in wild-type egg chambers. In contrast to tracheal cells, we detected no Ple protein in wild-type egg chambers (FIG. 3A). The antibody was effective because we could detect Ple ectopically expressed using c306Gal4 (FIG. 3B), as well as endogenous expression of Ple in neurons in the adult brain⁵⁵ (FIG. 3C). Therefore, it is unlikely that negative regulation of Ple activity is the key function of Catsup in border cells, suggesting that its role in border cell migration is distinct from its role in tracheal development.

In wing imaginal disc epithelia, multiple transmembrane receptor proteins, including Notch and EGFR, accumulate abnormally in Catsup mutant cells⁴⁰. We similarly found abnormal intracellular accumulation of Notch in follicle cells generally (FIG. 3D, D′) and border cells specifically (FIG. 3E, E′) upon Catsup knockdown. Cells lacking Catsup also exhibited defective Notch transcriptional activity, detected by the Notch responsive element reporter⁵⁶ (FIG. 3F, F′). Since Notch signaling is essential for border cell migration, and expression of constitutively active Notch (the Notch intracellular domain, NICD), which does not require intracellular trafficking, ligand binding, or processing, rescues impaired Notch signaling in border cells⁵⁷, we asked whether NICD could rescue Catsup knockdown. However, neither NICD expression nor overexpression of the Notch specific chaperone O-fucosyltransferase-1⁵⁸ was sufficient to rescue Catsup RNAi. As in imaginal discs, EGFR also accumulated abnormally in Catsup RNAi-expressing border cells (FIG. 3G, G′) and epithelial follicle cells (FIG. 3H, H′), whereas E-Cadherin was unaffected (FIG. 31, I′). From these observations, we conclude that Catsup prevents intracellular accumulation of particular transmembrane proteins and promotes Notch signaling, migration and survival in multiple cell types and organisms.

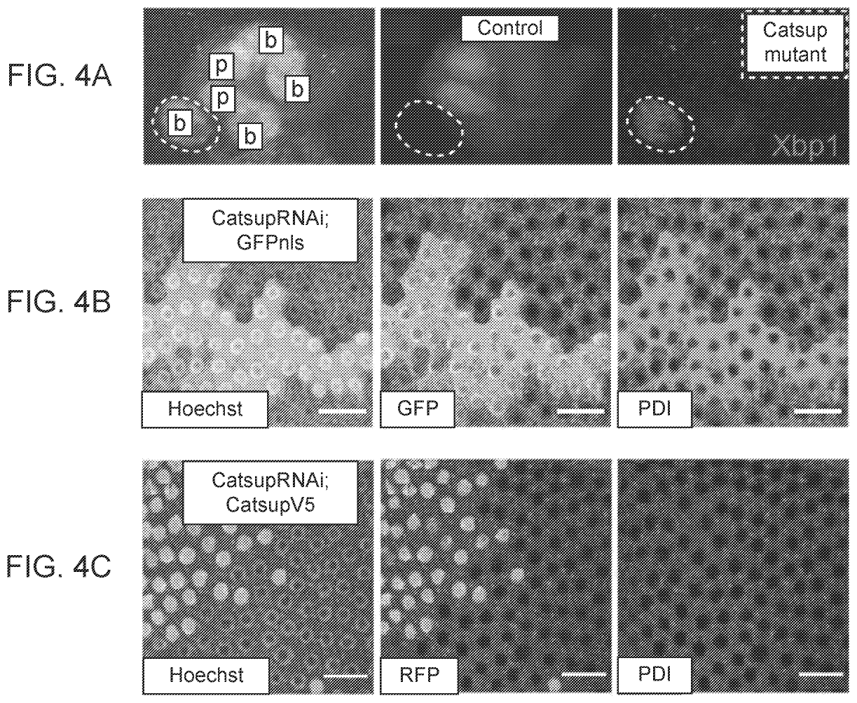

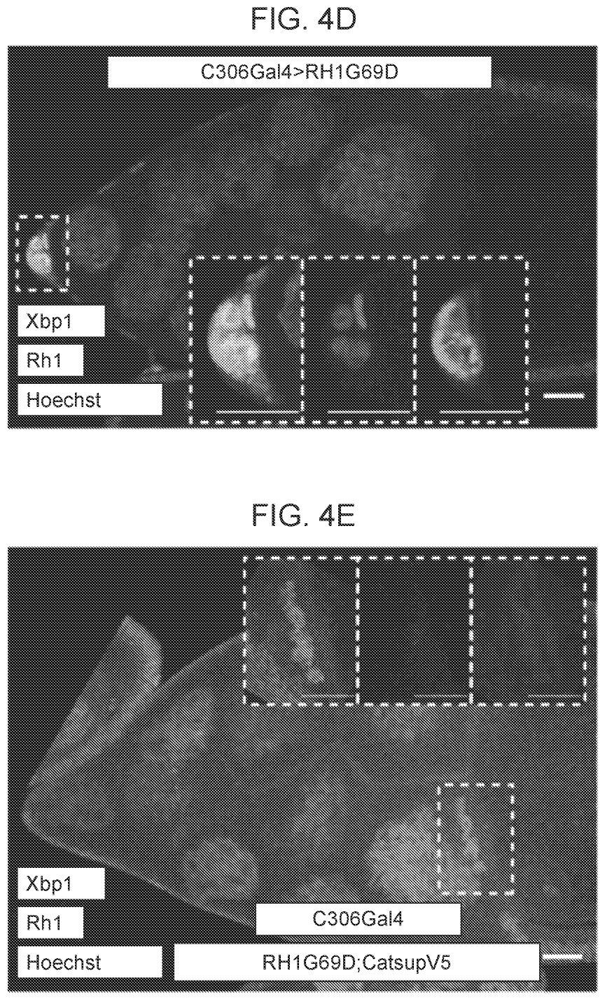

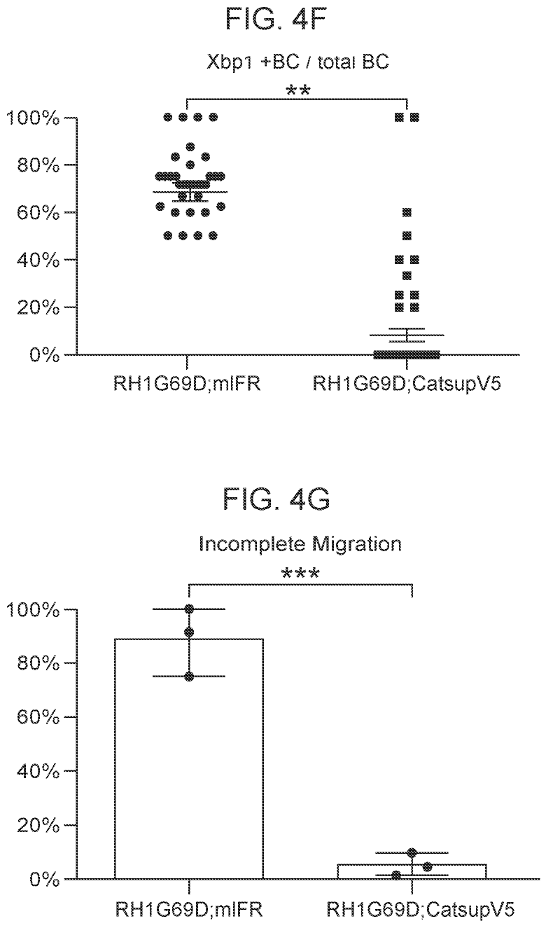

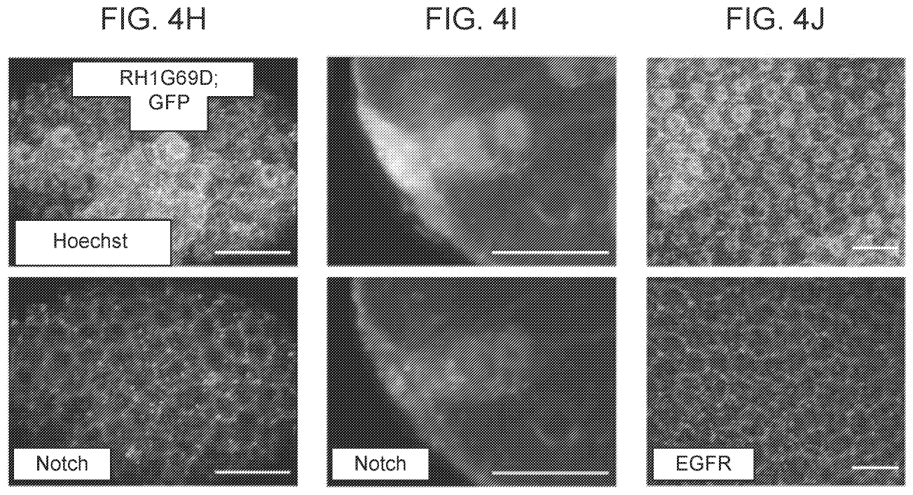

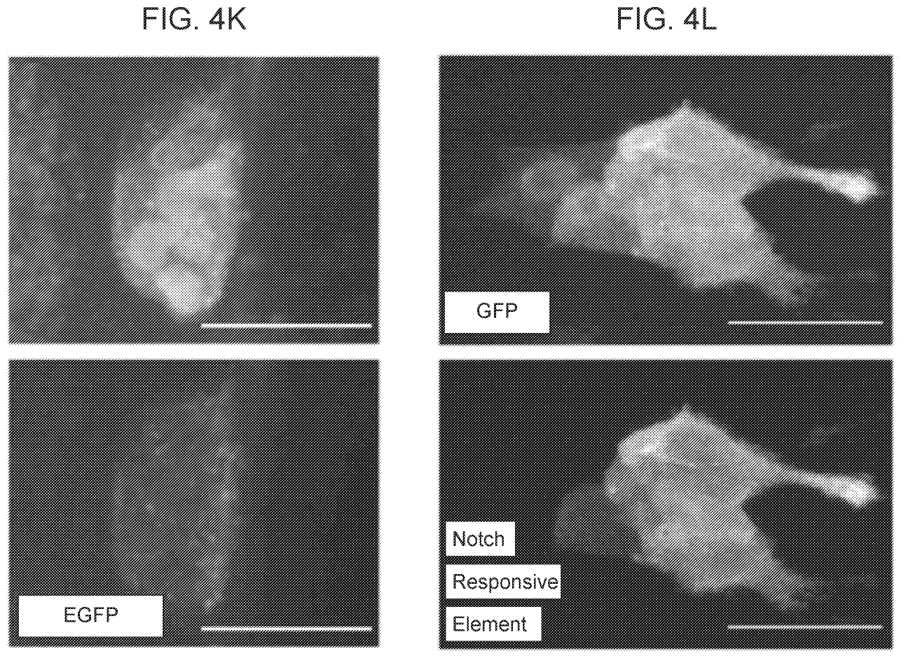

The abnormal intracellular accumulation of Notch and EGFR suggested possible disruption of ER protein-folding homeostasis (a condition commonly referred to as “ER stress”) and activation of the unfolded protein response (UPR). To test if Catsup knockdown induces the UPR in follicle cells, we compared expression of the ER stress marker Xbp1::EGFP⁵⁹ in mosaic border cell clusters composed of heterozygous Catsup^{+/-} and homozygous Catsup^{-/-} mutant cells. We found high levels of Xbp1 in Catsup^{-/-} but not Catsup^{+/-} border cells (FIG. 4A-A″). Consistent with this result, we observed increased expression of PDI (FIG. 4B-B″). Accumulation of XBP1 and PDI are indicative of induction of an adaptive UPR⁶⁰. This effect was rescued by co-expression of CatsupV5 (FIG. 4C-C″). These results show that cells lacking Catsup experience ER stress and impaired migration, suggesting that the ER stress itself might inhibit motility.

To test whether ER stress impairs border cell migration, we expressed a misfolded rhodopsin protein Rh1^{G69D}, known to induce ER stress⁶¹ As expected, Rh1^{G69D} induced Xbp1 expression in border cells (FIG. 4D insert). Notably, Rh1^{G69D} expression also blocked migration (FIG. 4D), showing that high levels of a misfolded protein in the ER and the ensuing ER stress are sufficient to inhibit motility. Since loss of Catsup caused ER stress, we wondered if Catsup over-expression might suppress ER stress. Interestingly, overexpressing CatsupV5 rescued Rh1^{G69D}-induced Xbp1 expression (FIGS. 4E, 4F) and border cell migration (FIGS. 4E and 4G). These results suggest that Catsup is a limiting factor for preventing ER stress, which hinders cell motility.

The UPR reinstates ER homeostasis by upregulating the protein-folding capacity of the ER and increasing its protein-degradation capacity⁶². During ERAD, misfolded proteins are extruded from the ER, ubiquitinated, and degraded by the proteasome⁶⁰. To test whether Catsup overexpression might enhance ERAD, we examined Rh1^{G69D} protein abundance in cells over-expressing Catsup. Catsup overexpression reduced Rh1^{G69D} protein to an undetectable level (FIGS. 4D, 4E) suggesting that Catsup function is limiting for ERAD.

To distinguish whether ER stress disrupts trafficking of Notch and EGFR, we examined Notch and EGFR abundance and localization in Rh1^{G9D}-expressing cells. Notch abundance and localization were normal in Rh1^{G69D}-expressing epithelial follicle cells (FIG. 4H, H′) and border cells (FIG. 4I, I′) as were EGFR expression and localization (FIGS. 4J-4K′). This result suggests that ER stress per se does not disrupt Notch or EGFR trafficking, which has previously been suggested as an interpretation of the Catsup mutant phenotype.

Interestingly, despite normal localization and abundance of Notch in these cells, Notch signaling was nevertheless impaired (FIG. 4L, L′). Thus, ER stress induced by accumulation of a misfolded ER client protein does not affect Notch or EGFR proteostasis but does impair Notch transcriptional activity. This finding is consistent with an earlier study that identified a pharmacological inhibitor of ZIP7 in a screen for compounds that block transcriptional responses to over-expressed NICD⁶³, which does not require Notch trafficking through the ER, cell surface expression, ligand binding, or proteolytic activation, although the authors concluded that ZIP7 promotes Notch trafficking⁶³. Our results imply that ER stress reduces NICD transcriptional activity, whether caused by Catsup mutation, Rh1^{G69D} expression, or ZIP7 pharmacological inhibition.

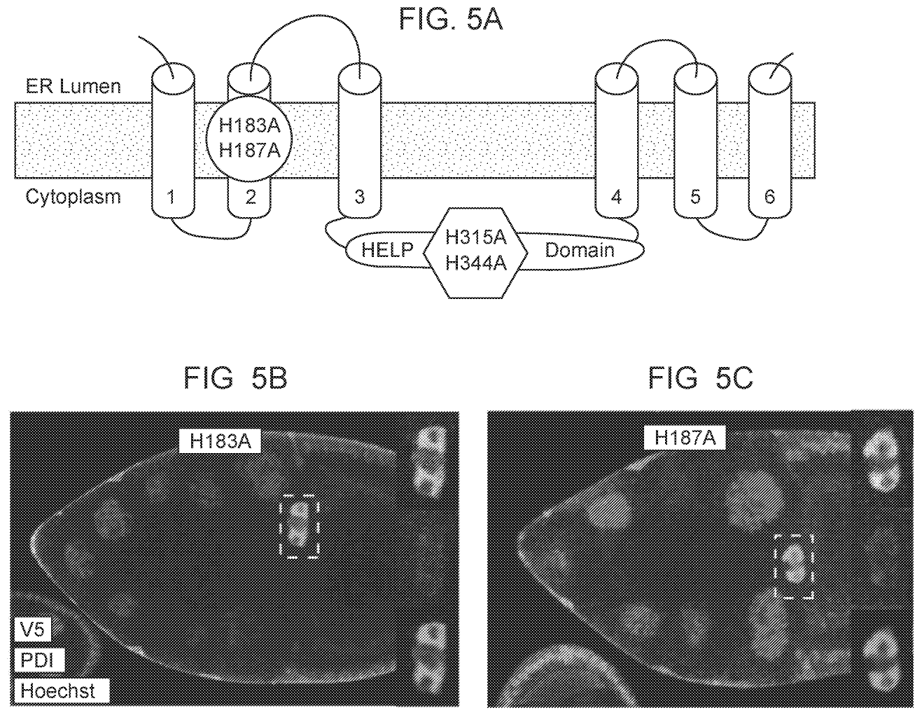

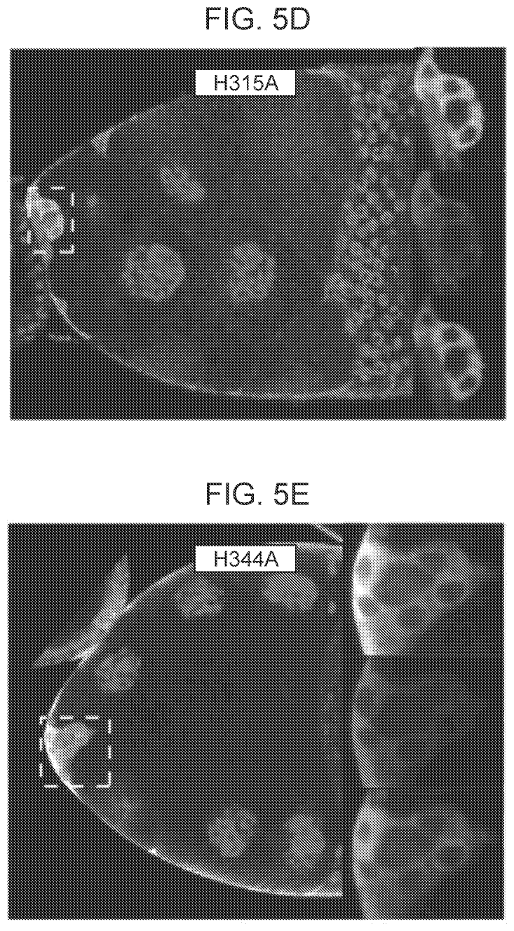

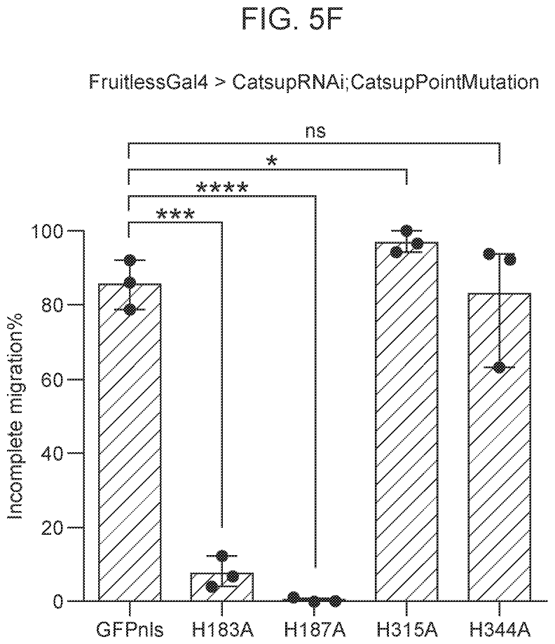

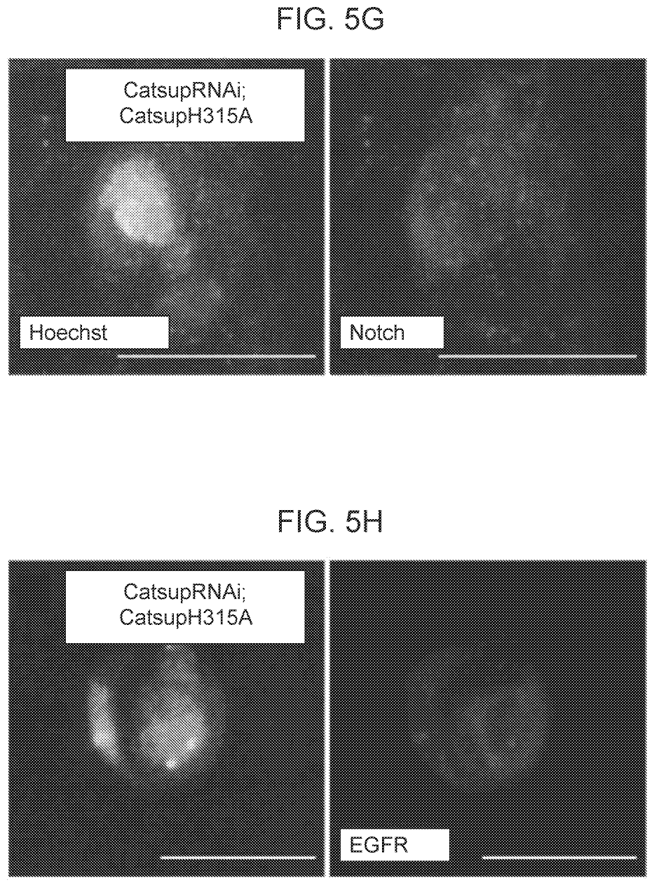

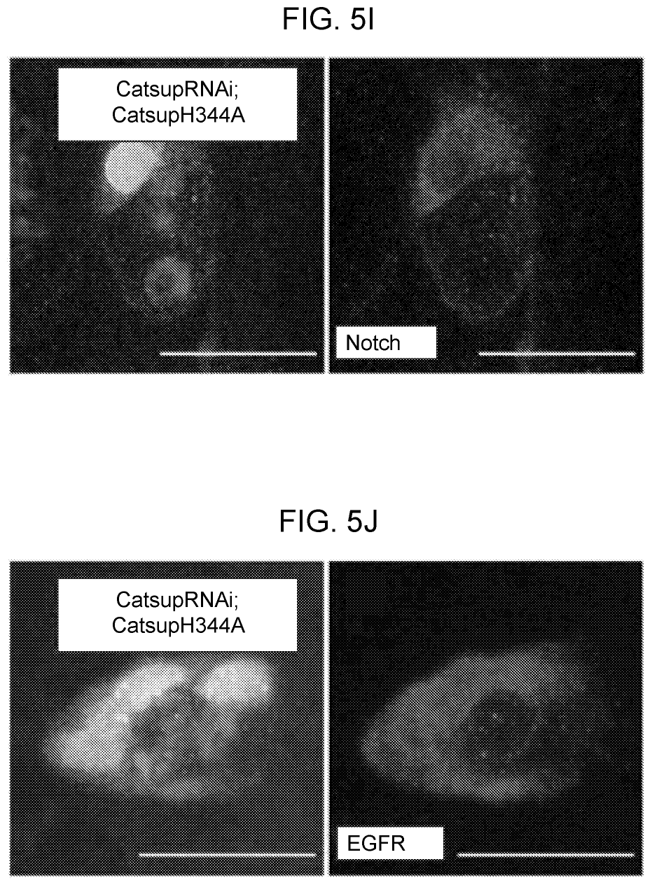

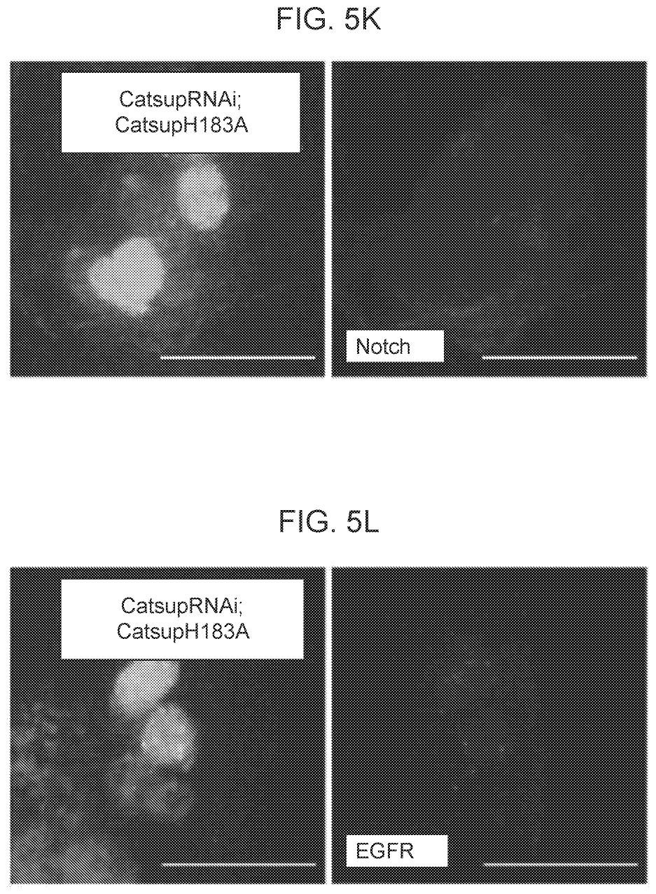

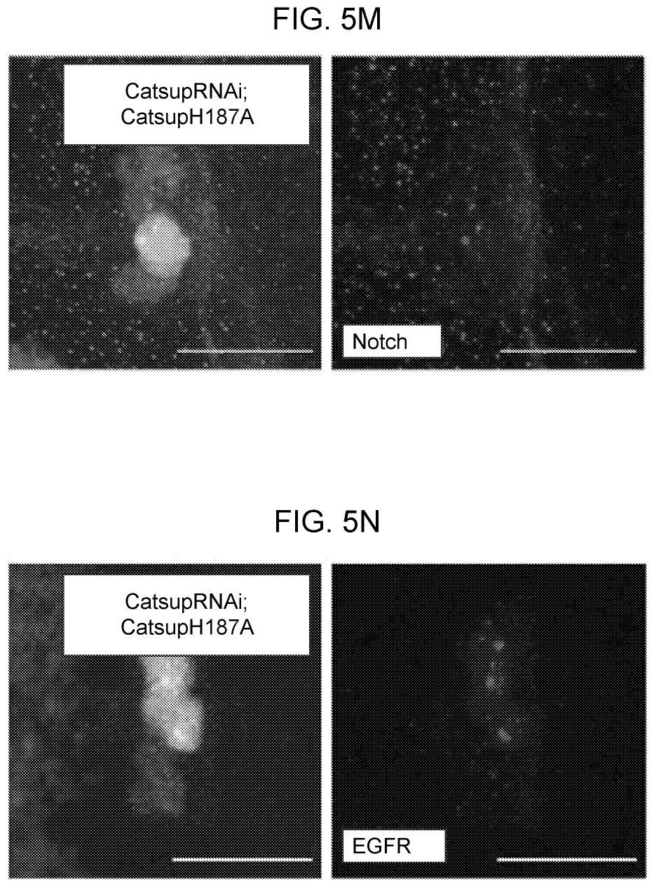

ZIP7 resides in the ER membrane and transports Zn²⁺ from the ER to the cytosol⁶⁴. To test whether the Zn²⁺ transporter activity of Catsup was important for border cell migration, we introduced point mutations, H315A and H344A, which replace histidine residues that are required for Zn²⁺ transport and are conserved between Catsup, ZIP7 and a more distant family member from Arabidopsis IRT1. As controls, we engineered Catsup^{H187A} and Catsup^{H183A} mutants that do not affect Zn²⁺ transport in IRT1⁶⁵. We generated transgenic flies expressing the mutants under Gal4/UAS control and included a V5 tag so that we could monitor protein abundance and localization. We then co-expressed each of these RNAi-resistant transgenes with CatsupRNAi and evaluated protein expression and border cell migration (FIGS. 5B-5E). The point mutations that do not disrupt Zn²⁺ transport, Catsup^{H187A} and Catsup^{H183A} were able to rescue border cell migration to nearly wild type levels (FIG. 5F) whereas neither Castup^{H344A} nor Catsup^{H315A} provided significant rescue (FIG. 5F). All the proteins were stably expressed and correctly localized to the ER (FIGS. 5B-5E), therefore the lack of rescue was likely a consequence of impaired transporter activity rather than impaired expression, localization, or another function. Similarly, the Zn²⁺-transport-deficient proteins failed to rescue accumulation of Notch and EGFR (FIGS. 5G-5J′) whereas Catsup^{H187A} and Catsup^{H183A} (FIGS. 5K-5N′) did. From these experiments, we conclude that Zn²⁺ transport is an essential function of Catsup in promoting ERAD.

### Discussion

### A Model for Catsup Function: Local Zn²⁺ Transport is Limiting for ERAD and Mitigation of ER Stress

In this study we explored the roles of the multifunctional protein Catsup in the Drosophila ovary. Catsup is a conserved protein that goes by names including ZRT1 in yeast, IRT1 in plants, and SLC39a7/Zip7/Ke4 in mammals. Prior to this work, diverse functions have been attributed to ZIP7 orthologs at the biochemical, cellular, tissue, and organ levels. Integrating prior studies with the results presented here, we propose a working model for a conserved function for Catsup in ERAD (FIG. 50) which mitigates ER stress and promotes cell migration and survival.

ERAD requires a complex machinery involving dozens of proteins responsible for recognizing misfolded proteins in the ER, extruding them to the cytoplasm by retrotranslocation, ubiquitinating them, and degrading them via the proteasome⁶⁶. ERAD E3 RING finger ubiquitin ligases, which reside in the ER membrane and require cytoplasmic Zn²⁺ for their catalytic activity, are crucial components of this system⁶⁶. There is little free Zn²⁺ in cells because most Zn²⁺ is bound to proteins⁶⁷. Cytosolic concentration estimates range from 5-1,000 pM, which are orders of magnitude lower than free Ca²⁺. Thus, an appealing possibility is that Catsup provides an essential, local source of Zn²⁺ at the ER/cytosol interface for ERAD E3 ubiquitin ligases. Consistent with this idea, overexpression of either of two ERAD E3 ubiquitin ligases, SORDD1/2, suppresses the proteotoxic effects of Rh1^{G69D} expression in the Drosophila eye⁶⁸, just as Catsup overexpression reduced the levels of misfolded Rh1^{G69D} protein expressed ectopically in border cells and alleviated all of the associated phenotypes including ER stress and border cell migration defects. This similarity between Catsup/ZIP7 and ERAD E3 ubiquitin ligase overexpression in rescuing Rh1^{G69D} phenotypes supports the idea that they function in a common pathway.

The abnormal accumulation of Notch and EGFR in Catsup mutant border cells resembles the Catsup phenotype in Drosophila wing imaginal discs. Although Notch and EGFR regulate cell fate in imaginal disc cells and migration in border cells, our results support a common role for Catsup in both tissues. Using live cell labeling with an antibody against the extracellular domain, Groth et al⁴⁰ showed that normal levels of Notch receptor protein are present on the plasma membrane of Catsup mutant cells and are endocytosed normally, despite abnormal accumulation in the ER. This result is more consistent with the role we propose for Catsup in ERAD than it is with the conclusion drawn by others that Catsup/ZIP7 affects Notch trafficking through the secretory pathway. An intriguing implication of the idea that Catsup/ZIP7 might provide a local source of Zn²⁺ for ERAD E3 ubiquitin ligases is that other Zn²⁺ transporters might also specialize in providing local Zn²⁺ for specific protein partners rather than, or in addition to, regulating global free Zn²⁺ within the cytosol or specific organelles, which is primarily how Zn²⁺ transport has been understood. Such a scenario could explain the need for 24 Zn²⁺ transporters in humans.

Our observations also raise the interesting question of why some proteins, like Notch and EGFR, are more prone to accumulation in the ER in Catsup mutant cells than others that also traverse the secretory pathway, such as E-cadherin. All of these are single-pass transmembrane proteins. Notch is a particularly large protein with 36 EGF-like repeats in and three cysteine-rich LIN12/Notch repeats in its extracellular domain, all of which require multiple disulfide bonds. Thus, Notch may be particularly prone to misfolding. By contrast, the EGFR extracellular domain is not as large or complex but, like Notch, it does contain two cysteine-rich domains and multiple disulfide bonds⁶⁹. In addition to effects on ERAD, it is possible that Catsup/ZIP loss-of-function causes excess Zn²⁺ to accumulate in the ER, which could in principle interfere with protein folding in the ER lumen. For example, excess Zn²⁺ might interact with cysteine residues, disrupting proper disulfide bond formation. In this case, Catsup mutant cells might be particularly prone to misfolding of newly synthesized proteins in the ER. Nolin et al. measured a rapid 2.5-fold increase in free Zn²⁺ in the ER lumen upon inhibition of ZIP7⁶³. Whether such a modest increase of free Zn²⁺ in the ER lumen would be sufficient to interfere with protein folding is not known.

Our results show that ER stress impairs Notch signaling independent of aberrant protein accumulation because Rh1^{G69D} induces ER stress and inhibits Notch signaling without abnormal Notch or EGFR protein accumulation in the ER lumen. How ER stress or the UPR inhibit Notch signaling is not clear, but the observation that a pharmacological inhibitor of ZIP7 was identified as an inhibitor of Notch signaling by NICD in cultured U2OS cells⁶³ suggests that there is a remarkably conserved requirement for Catsup/ZIP7 for Notch transcriptional activity. Nolin et al⁶³ showed that ZIP7 inhibition leads to accumulation of full length Notch and a decrease in the NICD, and concluded that Notch activation by proteolysis was perturbed upon inhibition of ZIP7. An alternative interpretation is that full-length Notch accumulates in the ER lumen due to inhibition of ERAD, and that NICD is, independently, degraded more rapidly as part of the global ER stress response.

The ability of Catsup overexpression to alleviate the ER stress and cellular defects due to Rh1^{G69D} expression has some general biomedical implications. Dominant mutations in opsins are the most common cause of retinal degeneration in human patients, for which there is currently no effective prevention or therapy. Ectopic expression of proteins that enhance ERAD may be a new therapeutic strategy to consider. Additionally, toxic protein aggregates have been proposed to kill neurons by inhibiting ERAD in numerous neurodegenerative diseases including Huntington's, Alzheimer's, Parkinson's, frontotemporal dementia, and others, even when the toxic protein is not localized in the ER⁷⁰. Thus, strategies to enhance ERAD may be useful in treating these diseases as well.

The suppression of ER stress and border cell migration by Catsup overexpression is consistent with the observation that ZIP7 is over-expressed in numerous cancers where it promotes survival, proliferation and migration and correlates with disease progression, invasion, and metastasis. The similarities in Catsup/ZIP7 functions and phenotypes across disparate cells, tissues, and organisms suggests that the border cell system offers an excellent model for deciphering the fundamental and conserved effects of this protein in vivo.

### Materials and Methods

### Drosophila Genetics

Catsup mutant fly was generated by ethyl methanesulfonate (EMS) mutagen³⁷. The mutation results in glycine(G) to aspartic acid(D) replacement at amino acid 178. The FLP/FRT system was used to generate the Catsup^{G178D} homozygous mutant clones by combining FRT40A-Catsup^{G178D} with hsFLP12,yw;ubi:GFPnls, FRT40A or hsFLP12,yw;ubi:RFPnls, FRT40A/(CyO). Catsup::GFP expression pattern visualized by the line from VDRC 318542 in the fTRG stocks library. The UAS-CatsupRNAi transgenic line is from VDRC 100095 P{KK103630}VIE-260B. Wild type rescue w[*]; sna[Sco]/CyO; P{w[+mCJ=UAS-Catsup. V5}6 Bloomington 63229. Additional transgenic Drosophila stocks used: UAS-wRNAi/Cyo is a lab stock, UAS-PleRNAi Bloomington 25796 y[1] v[1]; P{y[+t7.7] v[+t1.8]=TRiP.JF01813}attP2, UAS-Ple is Bloomington 37539 w[*]; P{w[+mC]=UAS-ple.T}331f2, O-fucosyltransferasel Bloomington 9376 P{UAS-O-fut.}]1.1, UAS-Notch.Intracellular.Domain on third chromosome was a gift from Artavanis-Tsakonas lab⁷¹. The ER stress marker UAS-Xbp1-EGFP.HG Bloomington 60731 w[*]; P{w[+mC]=UAS-Xbp1.EGFP.HG}3, UAS-HSC70-3 Bloomington 5843 w[126]; P{w[+mC]=UAS-Hsc70-3. WT}B. Catsup point mutations were cloned into vector pUASt-attb with forward primer ctctgaatagggaattgggATGGCCAAACAAGTGGCTGA (SEQ ID NO:1) and reverse primer ccgcagatctgttaacgtcaCGTAGAATCGAGACCGAGGAGAG (SEQ ID NO:2). The vector was injected to attp2 flies y1 w67c23; P{CaryP}attP2 by BestGene Inc.

### Design of UAS-RNAi-Resistant Catsup Point Mutations

When generating UAS-Catsup-point-mutations, we designed the construct so it cannot be targeted by the CatsupRNAi sequences by substituting redundant codons for the same amino acids within the region targeted by the RNAi. The RNAi resistant sequence is below with nucleic acid substitutions in lower case:

(SEQ ID NO: 3)
ACAcGGcCAttcCCAtGAcATGtcCATcGGctTGTGGGTgCTgGGcGGcATtATcGCgTTtCTgag
cGTcGAaAAgtTGGTgCGtATcCTgAAaGGaGGcCAcGGcGGcCAtGGaCAttcCCAcGGcGCc
CCcAAaCCcAAgCCcGTcCCcGCcAAaAAgAAaagCagcGAtAAgGAgGAttcCGGcGAcGGcG
AtAAgCCcGCcAAaCCcGCgAAaATtAAaagCAAaAAgCCcGAgGCcGAaCCcGAgGGaGAgG
TcGAaATcagCGGaTAtcTGAAccTGGCcGCcGAtTTcGCcCAtAAtTTtACgGAcGGatTGGCg
ATtGGaGCgagCTAccTGGCcGGaAAttcCATcGGaATcGTcACgACcATtACcATctTGtTGCAt
GAgGTcCCgCAcGAaATcGGcGAtTTcGCgATcCTgATcAAaagcGGaTGcagCcGcCGcAAaG
CcATGCTgtTGCAaCTgGTgACcGCcCTgGGcGCccTGGCcGGaACcGCcCTgGCcCTgtTGGG
cGCcGGcGGaGGcGAtGGcagcGCgCCcTGGGTgcTGCCgTTtACcGCgGGaGGcTTcATcTAt
ATtGCcACcGTcAGcGTgtTGCCcGAatTGCTgGAaGAaagcACcAAgtTGAAgCAaagctTGAAa
GAgATtTTcGCctTGCTgACCGGCGTAGCCCTAATGATCGTTATCGCCAAGTTCGAGGg.

Point mutations were designed by changing codons at the following site CatsupH183A (CAC to GCC), CatsupH187A (CAT to GCT), CatsupH315A (CAT to GCT), CatsupH344A (GCT to CAT).

### Immunostaining and Confocal Imaging

Female flies were fattened with yeast for 2 days at 29° C. Egg chambers were dissected from ovaries of female flies in Schneider's medium with 10% FBS (pH=6.85-6.95) as described previously⁷². Freshly dissected egg chambers were fixed in 4% paraformaldehyde and then incubated overnight in 1×PBS with 0.4% triton and the following primary antibodies: mouse anti-PDI (1:200) (ADI-SPA-891-D Enzo Life Sciences, Inc.), chicken anti-GFP (1:200) (ab13970 Abcam plc.), Ple (anti-TH) antibody (a gift from the Craig Montell lab), mouse anti-Notch intracellular domain (1:100) C17.9C6 DSHB, rat Ecadherin antibody DCAD2 (1:50) DSHB, V5 Tag Monoclonal Antibody-Alexa Fluor 555 (2F11F7) Invitrogen, mouse anti-dEGFR (1:2000) E2906 Sigma Aldrich. O-fut1 antibody was used to confirm O-fut1 overexpression, and was a gift from Kenneth D. Irvine lab 73. Secondary antibodies were incubated for 2 hours, together with Hoechst stain for nuclei, and phalloidin stain for F-actin. Mouse anti-PDI and mouse anti-V5-555 co-staining was done by first staining with PDI primary and secondary, followed by a thorough washout, and application of anti-V5-555 overnight. Immunostained samples were mounted in VECTASHIELD mounting medium from Vector Laboratories. Zeiss LSM780 and LSM800 confocal microscopes were used to acquire images. Images were processed using FIJI, rotated and cropped for presentation.

### Sequence Alignment

Catsup and ZIP7 amino acid sequences were acquired from NCBI in a FASTA format. The files were input into T-coffee tcoffee.crg.cat/apps/tcoffee/do:regular to generate multiple sequence alignment. The output was fed into Boxshade ch.embnet.org/software/BOX_form.html to generate the sequence alignment with black and grey shades to show the conserved sequence region.

### BIBLIOGRAPHY

1. Mishra, A. K., Campanale, J. P., Mondo, J. A. & Montell, D. J. Cell interactions in collective cell migration. Development 146, (2019).
2. Friedl, P. & Mayor, R. Tuning Collective Cell Migration by Cell-Cell Junction Regulation. Cold Spring Harb. Perspect. Biol. 9, (2017).
3. Friedl, P. & Gilmour, D. Collective cell migration in morphogenesis, regeneration and cancer. Nat. Rev. Mol. Cell Biol. 10, 445-457 (2009).
4. Friedl, P., Locker, J., Sahai, E. & Segall, J. E. Classifying collective cancer cell invasion. Nat. Cell Biol. 14, 777-783 (2012).
5. Silver, D. L. & Montell, D. J. Paracrine signaling through the JAK/STAT pathway activates invasive behavior of ovarian epithelial cells in Drosophila. Cell 107, 831-841 (2001).
6. Bai, J. & Montell, D. Eyes absent, a key repressor of polar cell fate during Drosophila oogenesis. Development 129, 5377-5388 (2002).
7. Jang, A. C.-C., Chang, Y.-C., Bai, J. & Montell, D. Border-cell migration requires integration of spatial and temporal signals by the BTB protein Abrupt. Nat. Cell Biol. 11, 569-579 (2009).
8. Bai, J., Uehara, Y. & Montell, D. J. Regulation of invasive cell behavior by taiman, a Drosophila protein related to AIB1, a steroid receptor coactivator amplified in breast cancer. Cell 103, 1047-1058 (2000).
9. McDonald, J. A., Pinheiro, E. M. & Montell, D. J. PVF1, a PDGF/VEGF homolog, is sufficient to guide border cells and interacts genetically with Taiman. Development 130, 3469-3478 (2003).
10. McDonald, J. A., Pinheiro, E. M., Kadlec, L., Schupbach, T. & Montell, D. J. Multiple EGFR ligands participate in guiding migrating border cells. Dev. Biol. 296, 94-103 (2006).
11. Dai, W. et al. Tissue topography steers migrating Drosophila border cells. BioRxiv (2020) doi:10.1101/2020.09.27.316117.
12. Murphy, A. M. & Montell, D. J. Cell type-specific roles for Cdc42, Rac, and RhoL in Drosophila oogenesis. J. Cell Biol. 133, 617-630 (1996).
13. Kim, J. H. et al. Psidin, a conserved protein that regulates protrusion dynamics and cell migration. Genes Dev. 25, 730-741 (2011).
14. Lee, T., Feig, L. & Montell, D. J. Two distinct roles for Ras in a developmentally regulated cell migration. Development 122, 409-418 (1996).
15. Cai, D. et al. Mechanical feedback through E-cadherin promotes direction sensing during collective cell migration. Cell 157, 1146-1159 (2014).
16. McDonald, J. A., Khodyakova, A., Aranjuez, G., Dudley, C. & Montell, D. J. PAR-1 kinase regulates epithelial detachment and directional protrusion of migrating border cells. Curr. Biol. 18, 1659-1667 (2008).
17. Wang, X., He, L., Wu, Y. I., Hahn, K. M. & Montell, D. J. Light-mediated activation reveals a key role for Rac in collective guidance of cell movement in vivo. Nat. Cell Biol. 12, 591-597 (2010).
18. Duchek, P. & Rorth, P. Guidance of cell migration by EGF receptor signaling during Drosophila oogenesis. Science 291, 131-133 (2001).
19. Fulga, T. A. & Rorth, P. Invasive cell migration is initiated by guided growth of long cellular extensions. Nat. Cell Biol. 4, 715-719 (2002).
20. Ramel, D., Wang, X., Laflamme, C., Montell, D. J. & Emery, G. Rab11 regulates cell-cell communication during collective cell movements. Nat. Cell Biol. 15, 317-324 (2013).
21. Assaker, G., Ramel, D., Wculek, S. K., Gonziláz-Gaitán, M. & Emery, G. Spatial restriction of receptor tyrosine kinase activity through a polarized endocytic cycle controls border cell migration. Proc Natl Acad Sci USA 107, 22558-22563 (2010).
22. Miao, G., Godt, D. & Montell, D. J. Integration of Migratory Cells into a New Site In Vivo Requires Channel-Independent Functions of Innexins on Microtubules. Dev. Cell 54, 501-515.e9 (2020).
23. Colombié, N. et al. Non-autonomous role of Cdc42 in cell-cell communication during collective migration. Dev. Biol. 423, 12-18 (2017).
24. Chen, Y. et al. Protein phosphatase 1 activity controls a balance between collective and single cell modes of migration. elife 9, (2020).
25. Laflamme, C. et al. Evi5 promotes collective cell migration through its Rab-GAP activity. J. Cell Biol. 198, 57-67 (2012).
26. Ogienko, A. A. et al. GAGA regulates border cell migration in drosophila. Int. J. Mol. Sci. 21, (2020).
27. Berez, A., Peercy, B. E. & Starz-Gaiano, M. Development and analysis of a quantitative mathematical model of bistability in the cross repression system between APT and SLBO within the JAK/STAT signaling pathway. Front. Physiol. 11, 803 (2020).
28. Wang, X. et al. Temporal Coordination of Collective Migration and Lumen Formation by Antagonism between Two Nuclear Receptors. iScience 23, 101335 (2020).
29. Wang, H., Guo, X., Wang, X., Wang, X. & Chen, J. Supracellular Actomyosin Mediates Cell-Cell Communication and Shapes Collective Migratory Morphology. iScience 23, 101204 (2020).
30. Fox, E. F., Lamb, M. C., Mellentine, S. Q. & Tootle, T. L. Prostaglandins regulate invasive, collective border cell migration. Mol. Biol. Cell 31, 1584-1594 (2020).
31. Plutoni, C. et al. Misshapen coordinates protrusion restriction and actomyosin contractility during collective cell migration. Nat. Commun. 10, 3940 (2019).
32. Zeledon, C., Sun, X., Plutoni, C. & Emery, G. The ArfGAP Drongo Promotes Actomyosin Contractility during Collective Cell Migration by Releasing Myosin Phosphatase from the Trailing Edge. Cell Rep. 28, 3238-3248.e3 (2019).
33. Lamb, M. C., Anliker, K. K. & Tootle, T. L. Fascin regulates protrusions and delamination to mediate invasive, collective cell migration in vivo. Dev. Dyn. 249, 961-982 (2020).
34. Ghiglione, C., Jouandin, P., Cerezo, D. & Noselli, S. The Drosophila insulin pathway controls Profilin expression and dynamic actin-rich protrusions during collective cell migration. Development 145, (2018).
35. Sharma, A. et al. Insulin signaling modulates border cell movement in Drosophila oogenesis. Development 145, (2018).
36. Liu, Y. & Montell, D. J. Identification of mutations that cause cell migration defects in mosaic clones. Development 126, 1869-1878 (1999).
37. Wang, X. et al. Analysis of cell migration using whole-genome expression profiling of migratory cells in the Drosophila ovary. Dev. Cell 10, 483-495 (2006).
38. Stathakis, D. G. et al. The catecholamines up (Catsup) protein of Drosophila melanogaster functions as a negative regulator of tyrosine hydroxylase activity. Genetics 153, 361-382 (1999).
39. Hsouna, A., Lawal, H. O., Izevbaye, I., Hsu, T. & O'Donnell, J. M. Drosophila dopamine synthesis pathway genes regulate tracheal morphogenesis. Dev. Biol. 308, 30-43 (2007).
40. Groth, C., Sasamura, T., Khanna, M. R., Whitley, M. & Fortini, M. E. Protein trafficking abnormalities in Drosophila tissues with impaired activity of the ZIP7 zinc transporter Catsup. Development 140, 3018-3027 (2013).
41. Kambe, T. Zinc Transport: Regulation, in Encyclopedia of inorganic and bioinorganic chemistry (ed. Scott, R. A.) 1-9 (John Wiley & Sons, Ltd, 2011). doi:10.1002/9781119951438.eibc2135.
42. Taylor, K. M., Morgan, H. E., Johnson, A. & Nicholson, R. I. Structure-function analysis of HKE4, a member of the new LIV-1 subfamily of zinc transporters. Biochem. J. 377, 131-139 (2004).
43. Kambe, T., Tsuji, T., Hashimoto, A. & Itsumura, N. The physiological, biochemical, and molecular roles of zinc transporters in zinc homeostasis and metabolism. Physiol. Rev. 95, 749-784 (2015).
44. Nguyen, T. S. L., Kohno, K. & Kimata, Y. Zinc depletion activates the endoplasmic reticulum-stress sensor Irel via pleiotropic mechanisms. Biosci. Biotechnol. Biochem. 77, 1337-1339 (2013).
45. Tan, S. et al. Over-expression of the MxIRT1 gene increases iron and zinc content in rice seeds. Transgenic Res. 24, 109-122 (2015).
46. Zhang, P. et al. An uncleaved signal peptide directs the Malus xiaojinensis iron transporter protein Mx IRT1 into the ER for the PM secretory pathway. Int. J. Mol. Sci. 15, 20413-20433 (2014).
47. Adulcikas, J., Norouzi, S., Bretag, L., Sohal, S. S. & Myers, S. The zinc transporter SLC39A7 (ZIP7) harbours a highly-conserved histidine-rich N-terminal region that potentially contributes to zinc homeostasis in the endoplasmic reticulum. Comput. Biol. Med. 100, 196-202 (2018).
48. Tuncay, E. et al. Hyperglycemia-Induced Changes in ZIP7 and ZnT7 Expression Cause Zn2+ Release From the Sarco(endo)plasmic Reticulum and Mediate ER Stress in the Heart. Diabetes 66, 1346-1358 (2017).
49. Fauster, A. et al. Systematic genetic mapping of necroptosis identifies SLC39A7 as modulator of death receptor trafficking. Cell Death Differ. 26, 1138-1155 (2019).
50. Unable to find information for 1244995.
51. Borensztejn, A., Boissoneau, E., Fernandez, G., Agnès, F. & Pret, A.-M. JAK/STAT autocontrol of ligand-producing cell number through apoptosis. Development 140, 195-204 (2013).
52. Haas, P. & Gilmour, D. Chemokine signaling mediates self-organizing tissue migration in the zebrafish lateral line. Dev. Cell 10, 673-680 (2006).
53. Geisbrecht, E. R. & Montell, D. J. A role for Drosophila IAP1-mediated caspase inhibition in Rac-dependent cell migration. Cell 118, 111-125 (2004).
54. Wang, Z. et al. Catecholamines up integrates dopamine synthesis and synaptic trafficking. J. Neurochem. 119, 1294-1305 (2011).
55. Navarro, J. A. et al. Analysis of dopaminergic neuronal dysfunction in genetic and toxin-induced models of Parkinson's disease in Drosophila. J. Neurochem. 131, 369-382 (2014).
56. Terriente-Felix, A. et al. Notch cooperates with Lozenge/Runx to lock haemocytes into a differentiation programme. Development 140, 926-937 (2013).
57. Wang, X., Adam, J. C. & Montell, D. Spatially localized Kuzbanian required for specific activation of Notch during border cell migration. Dev. Biol. 301, 532-540 (2007).
58. Okajima, T., Xu, A., Lei, L. & Irvine, K. D. Chaperone activity of protein O-fucosyltransferase 1 promotes notch receptor folding. Science 307, 1599-1603 (2005).
59. Sone, M., Zeng, X., Larese, J. & Ryoo, H. D. A modified UPR stress sensing system reveals a novel tissue distribution of IRE1/XBP1 activity during normal Drosophila development. Cell Stress Chaperones 18, 307-319 (2013).
60. Karagoz, G. E., Aragón, T. & Acosta-Alvear, D. Recent advances in signal integration mechanisms in the unfolded protein response. [version 1; peer review: 2 approved]. F1000Res. 8, (2019).
61. Chow, C. Y., Avila, F. W., Clark, A. G. & Wolfner, M. F. Induction of excessive endoplasmic reticulum stress in the Drosophila male accessory gland results in infertility. PLoS ONE 10, e0119386 (2015).
62. Karagoz, G. E., Acosta-Alvear, D. & Walter, P. The Unfolded Protein Response: Detecting and Responding to Fluctuations in the Protein-Folding Capacity of the Endoplasmic Reticulum. Cold Spring Harb. Perspect. Biol. 11, (2019).
63. Nolin, E. et al. Discovery of a ZIP7 inhibitor from a Notch pathway screen. Nat. Chem. Biol. 15, 179-188 (2019).
64. Taylor, K. M., Hiscox, S., Nicholson, R. I., Hogstrand, C. & Kille, P. Protein kinase CK2 triggers cytosolic zinc signaling pathways by phosphorylation of zinc channel ZIP7. Sci. Signal. 5, ra11 (2012).
65. Rogers, E. E., Eide, D. J. & Guerinot, M. L. Altered selectivity in an Arabidopsis metal transporter. Proc Natl Acad Sci USA 97, 12356-12360 (2000).
66. Christianson, J. C. et al. Defining human ERAD networks through an integrative mapping strategy. Nat. Cell Biol. 14, 93-105 (2011).
67. Ohashi, W. et al. Zinc Transporter SLC39A7/ZIP7 Promotes Intestinal Epithelial Self-Renewal by Resolving ER Stress. PLoS Genet. 12, e1006349 (2016).
68. Xu, J., Zhao, H. & Wang, T. Suppression of retinal degeneration by two novel ERAD ubiquitin E3 ligases SORDD1/2 in Drosophila. PLoS Genet. 16, e1009172 (2020).
69. Ferguson, K. M. Structure-based view of epidermal growth factor receptor regulation. Annu. Rev. Biophys. 37, 353-373 (2008).
70. Shacham, T., Sharma, N. & Lederkremer, G. Z. Protein misfolding and ER stress in huntington's disease. Front. Mol. Biosci. 6, 20 (2019).
71. Go, M. J., Eastman, D. S. & Artavanis-Tsakonas, S. Cell proliferation control by Notch signaling in Drosophila development. Development 125, 2031-2040 (1998).
72. Dai, W. & Montell, D. J. Live imaging of border cell migration in drosophila. Methods Mol. Biol. 1407, 153-168 (2016).
73. Okajima, T., Xu, A. & Irvine, K. D. Modulation of notch-ligand binding by protein O-fucosyltransferase 1 and fringe. J. Biol. Chem. 278, 42340-42345 (2003).

### Example 2: Zip7 Prevents Rh1G69D-Induced Photoreceptor Cell Death

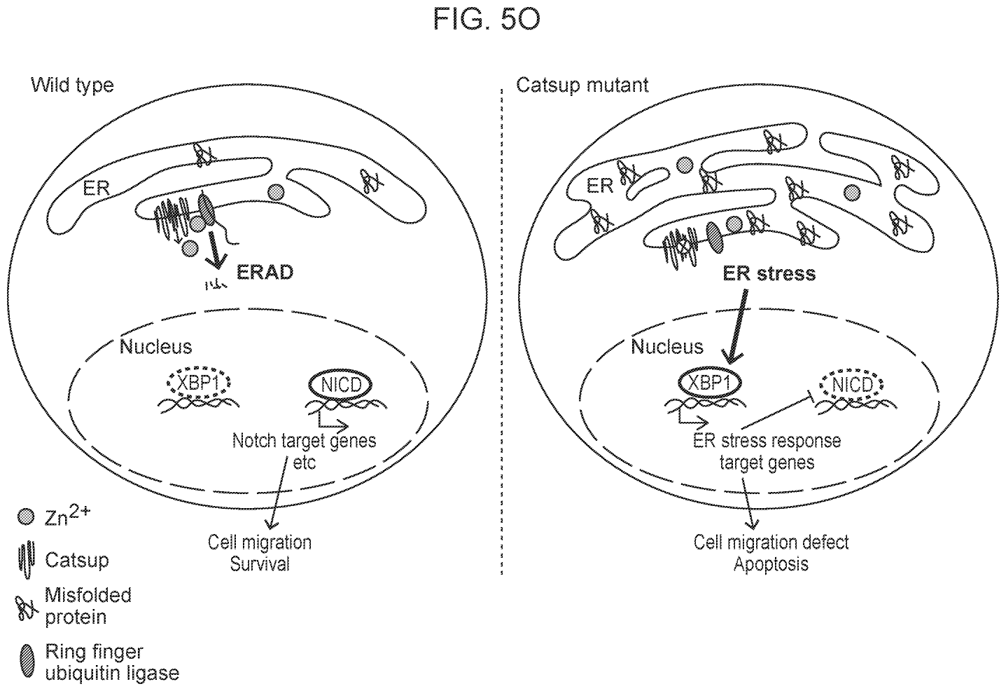

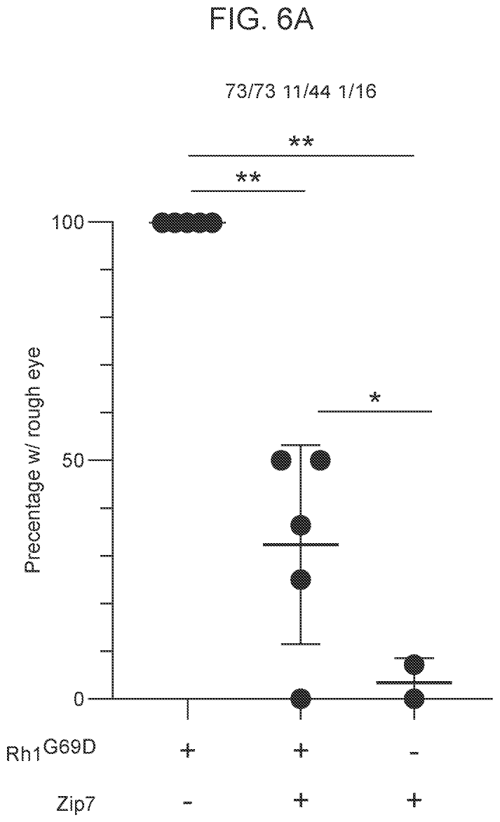

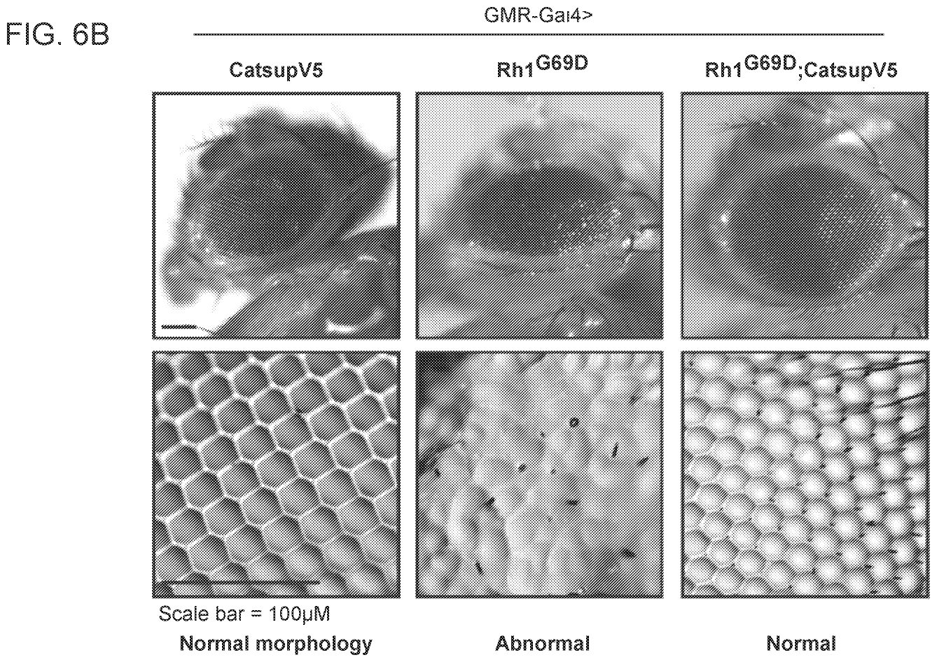

ZIP7 overexpression prevented photoreceptor cell death in the eyes of flies expressing mutant forms of rhodopsin. As shown in FIGS. 6A and 6B, ZIP7 prevented Rh1G69D-induced photoreceptor cell death, which causes a rough eye.

### Example 3: Zip7 Overexpression Prevents Neuronal Death in Flies Expressing Vapor 33 or Amyloid β42

Many neurodegenerative diseases involve abnormal protein aggregation that triggers the unfolded protein response (UPR). These include Alzheimer's disease (Ab42), Huntington's (mutant Htt), mutant alpha-synuclein (Parkinson's disease and other dementia), TDP-43 (amyotrophic lateral sclerosis (ALS) and other dementias), and VapB (ALS). ER stress, caused by these proteins, may be rescued by ZIP7 expression.

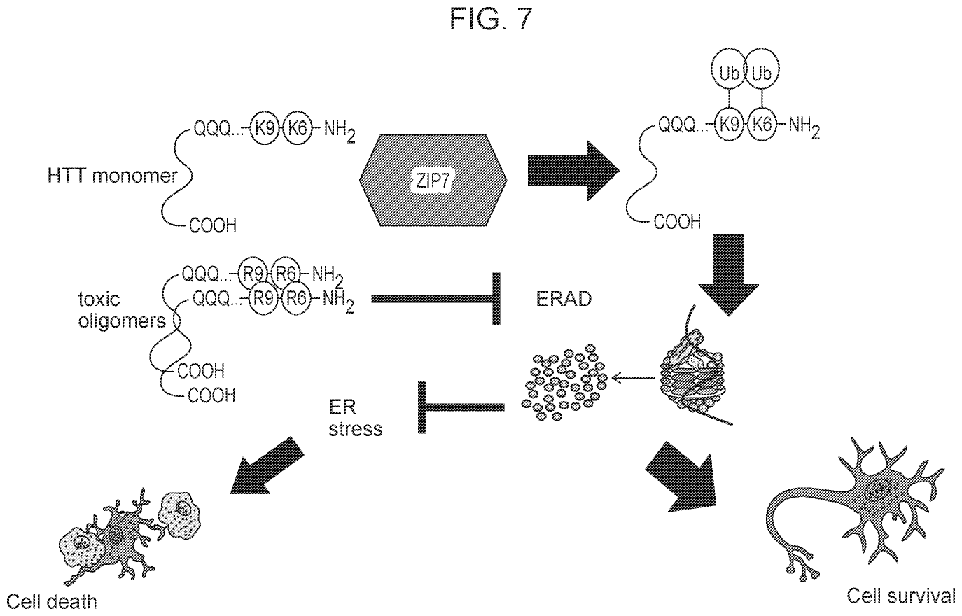

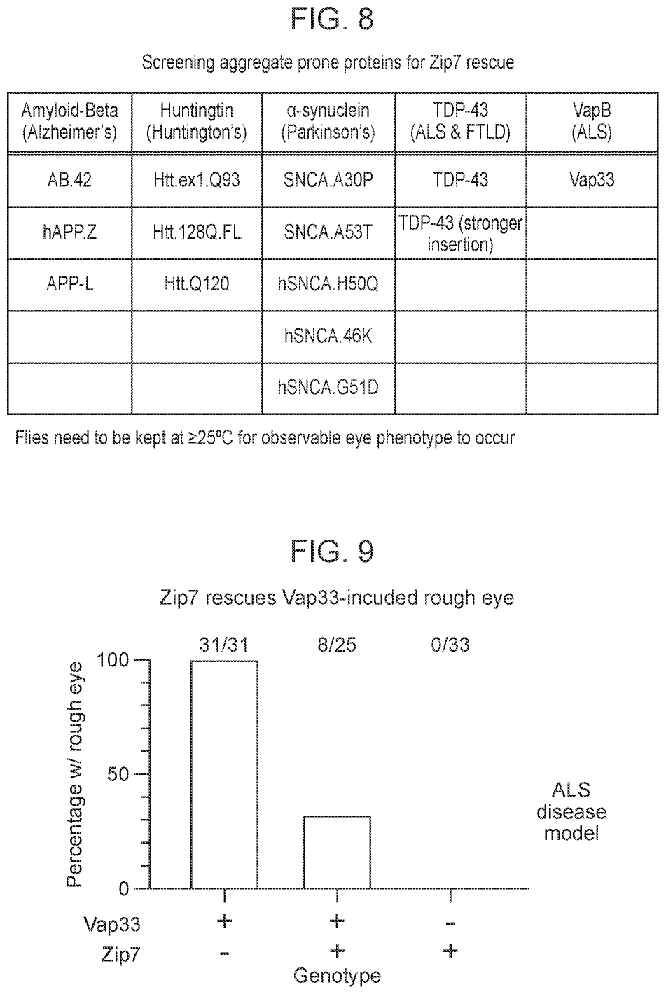

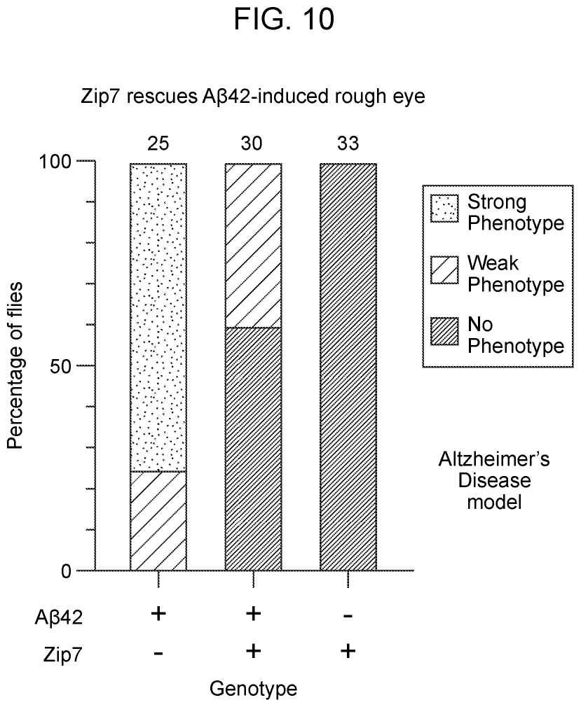

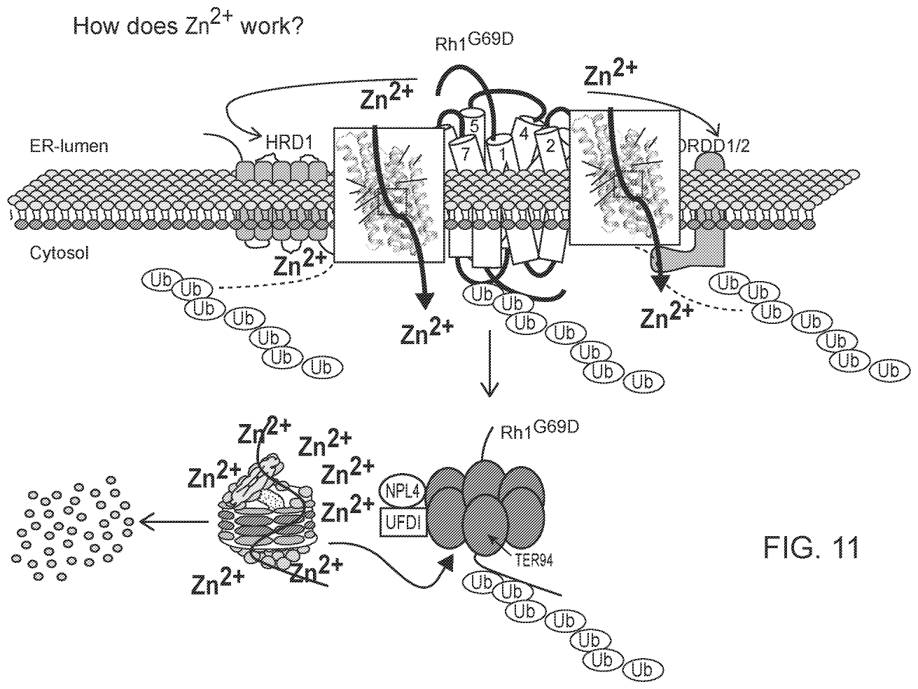

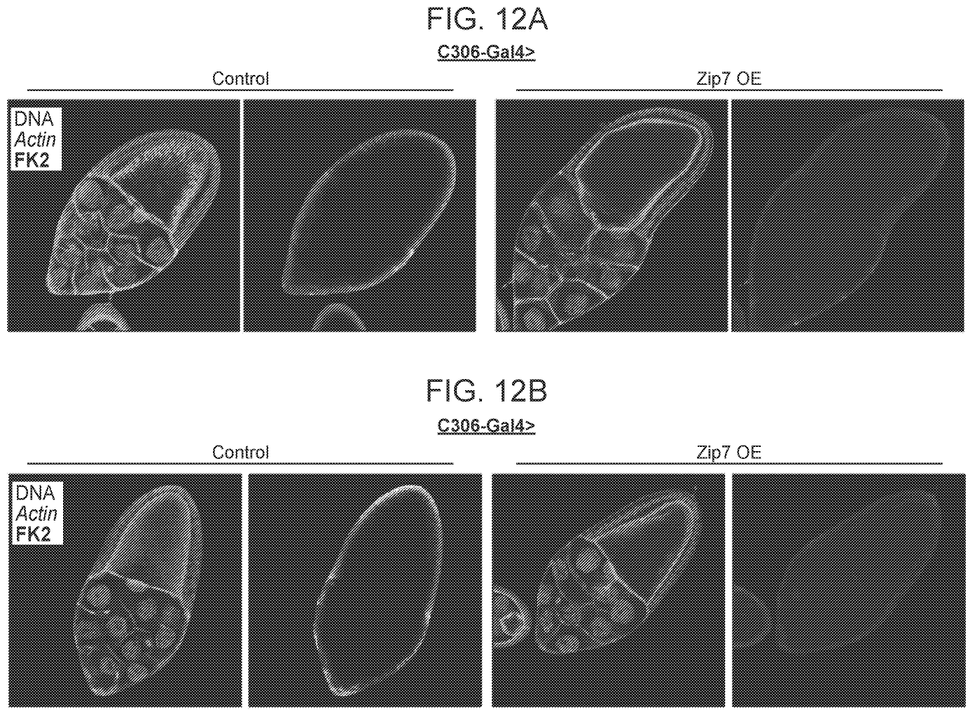

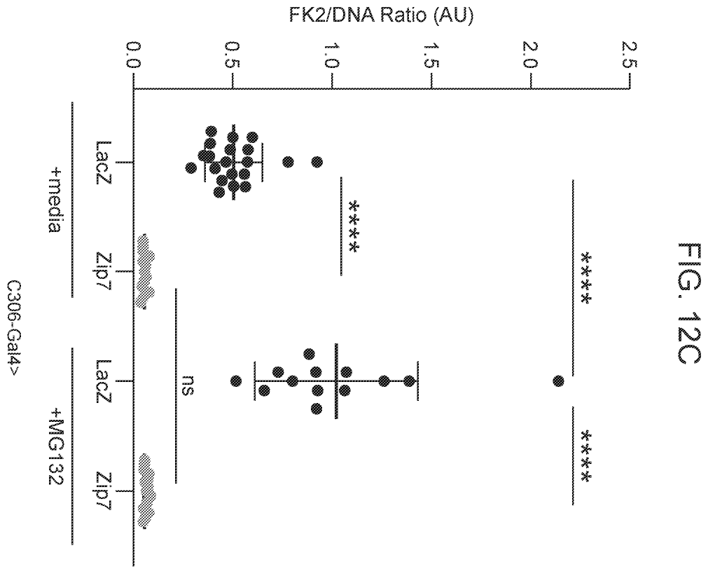

Overexpression of ZIP7 may mitigate ER stress and neuronal cell death in protein aggregation diseases (FIG. 7). We screened aggregate prone proteins for ZIP7 rescue (FIG. 8). ZIP7 overexpression suppressed neurodegeneration due to expression of Vap33 (FIG. 9) and amyloid β42 (FIG. 10).

### Example 4: Overexpressing Zip7 Decreases Ubiquitinated Protein Levels

Egg chambers with and without ZIP7 overexpression (OE) were incubated with media in the presence or absence of the MG132 proteasome inhibitor (10 μM) for 5 hours (FIGS. 12A and 12B). Overexpressing ZIP7 decreased ubiquitinated protein levels and the FK2/DNA ratio (FIG. 12C).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of providing a subject with zinc transporter protein 7 (ZIP7) to suppress pathological accumulation of a misfolded protein, the method comprising introducing an expression vector comprising a promoter operably linked to a coding sequence encoding the ZIP7 into a cell, wherein the cell expresses the ZIP in vivo in the subject in an effective amount sufficient to suppress the pathological accumulation of the misfolded protein in the cell.

2. The method of claim 1, wherein the cell is a retina cell or a brain cell.

3. The method of claim 2, wherein the retina cell is a photoreceptor cell.

4. The method of claim 2 or 3, wherein the misfolded protein is a misfolded rhodopsin protein.

5. The method of any one of claims 1-4, wherein the expression vector is introduced into the cell ex vivo or in vivo.

6. The method of any one of claims 1-5, wherein the subject has a disorder associated with protein misfolding.

7. The method of any one of claim 6, wherein the disorder associated with protein misfolding is retinitis pigmentosa.

8. The method of claim 6, wherein the disorder associated with protein misfolding is a neurodegenerative disease.

9. The method of any one of claims 1-8, wherein the misfolded protein is Rh1G69D rhodopsin, Vap33, or amyloid β42.

10. A method of treating a subject for a disorder associated with protein misfolding, the method comprising administering an expression vector comprising a promoter operably linked to a nucleotide sequence encoding zinc transporter protein 7 (ZIP7) to the subject, wherein the ZIP7 is expressed in vivo in the subject in a therapeutically effective amount sufficient to suppress pathological accumulation of the misfolded protein.

11. The method of claim 10, wherein the disorder associated with protein misfolding is retinitis pigmentosa or a neurodegenerative disease.

12. The method of claim 10 or 11, wherein the expression vector is administered locally into an eye or the brain of the subject.

13. The method of claim 10 or 11, wherein the expression vector is administered locally into a retina or a photoreceptor of the subject.

14. The method of any one of claims 10-13, wherein the misfolded protein is Rh1G69D rhodopsin, Vap33, or amyloid β42.

15. A method of treating a subject for a disorder associated with protein misfolding, the method comprising administering to the subject a therapeutically effective amount of a zinc transporter protein 7 (ZIP7) protein.

16. The method of claim 15, wherein the ZIP7 protein comprises or consists of an amino acid sequence having at least 90% identity to the sequence of SEQ ID NO: 5.

17. The method of claim 15 or 16, wherein the disorder associated with protein misfolding is retinitis pigmentosa or a neurodegenerative disease.

18. The method of any one of claims 15-17, wherein the ZIP7 protein is administered locally into an eye or the brain of the subject.

19. The method of any one of claims 15-18, wherein the ZIP7 protein is administered locally into a retina or a photoreceptor of the subject.

20. The method of any one of claims 15-19, wherein the ZIP7 protein is administered according to a daily dosing regimen or intermittently.

21. The method of any one of claims 15-20, wherein the misfolded protein is Rh1G69D rhodopsin, Vap33, or amyloid β42.

22. A method of enhancing endoplasmic reticulum (ER)-associated degradation (ERAD) or proteosome-associated degradation in a cell, the method comprising increasing expression or activity of zinc transporter protein 7 (ZIP7) in the cell.

23. The method of claim 22, wherein the cell is a retina cell.

24. The method of claim 23, wherein the retina cell is a photoreceptor cell.

25. The method of claim 23 or 24, wherein said increasing expression or activity of ZIP7 is sufficient to increase degradation of a misfolded rhodopsin protein and suppress accumulation of the misfolded rhodopsin protein in the retina cell.

26. The method of any one of claims 22-25, wherein said increasing expression of ZIP7 comprises transfecting the cell with a recombinant polynucleotide comprising a coding sequence encoding ZIP7.

27. The method of claim 26, wherein the recombinant polynucleotide is provided by a viral vector comprising a promoter operably linked to the coding sequence encoding the ZIP7.

28. The method of claim 27, wherein the coding sequence encoding the ZIP7 is integrated into a chromosomal locus of the transfected cell.

29. The method of claim 28, wherein an endogenous promoter is operably linked to the integrated coding sequence encoding the ZIP7 at the chromosomal locus.

30. A method of suppressing accumulation of a misfolded protein in an organ, cell, or tissue of a subject, the method comprising increasing expression or activity of zinc transporter protein 7 (ZIP7) in the organ, cell, or tissue.

31. The method of claim 30, wherein the organ is an eye or a brain.

32. The method of claim 30, wherein the tissue is neural tissue.

33. The method of claim 30, wherein the tissue is retina tissue.

34. The method of claim 30, wherein the cell is a retina cell.

35. The method of claim 34, wherein the retina cell is a photoreceptor cell.

36. The method of claim 35, wherein the misfolded protein is a rhodopsin protein.

37. The method of any one of claims 30-36, wherein the subject has retinitis pigmentosa.

38. The method of any one of claims 30-37, wherein the subject has a disorder associated with protein misfolding.

39. The method of claim 38, wherein the disorder associated with protein misfolding is a neurodegenerative disease.

40. The method of any one of claims 30-39, wherein the expression of ZIP7 is increased sufficiently to decrease pathological accumulation of a misfolded protein and increase cell survival.

41. The method of any one of claims 30-40, wherein said increasing expression of ZIP7 comprises providing a recombinant polynucleotide comprising a coding sequence encoding the ZIP7 to the organ, cell, or tissue, wherein the ZIP7 is expressed in the organ, cell, or tissue.

42. The method of claim 41, wherein the recombinant polynucleotide further comprises a viral vector comprising a promoter operably linked to the coding sequence encoding the ZIP7.

43. The method of claim 42, wherein the coding sequence encoding the ZIP7 is integrated into a chromosomal locus.

44. The method of claim 43, wherein an endogenous promoter is operably linked to the integrated coding sequence encoding the ZIP7 at the chromosomal locus.

45. The method of any one of claims 30-44, wherein the misfolded protein is Rh1G69D rhodopsin, Vap33, or amyloid β42.

46. A composition for use in a method of treating a disorder associated with protein misfolding, the composition comprising zinc transporter protein 7 (ZIP7) or an expression vector comprising a promoter operably linked to a coding sequence encoding ZIP7.

47. The composition of claim 46, further comprising a pharmaceutically acceptable excipient.
